(19) United States
(12) Patent Application Publication
(10) Pub. No.: US 2022/0401542 A1
(43) Pub. Date: Dec. 22, 2022

(54) MYCOBACTERIAL COMPOSITIONS AND BIOMARKERS FOR USE IN TREATMENT AND MONITORING OF THERAPEUTIC RESPONSIVENESS

(71) Applicant: Beth Israel Deaconess Medical Center, Inc., Boston, MA (US)

(72) Inventors: Dan H. BAROUCH, Newton, MA (US); Amanda J. MARTINOT, North Grafton, MA (US)

(73) Assignee: Beth Israel Deaconess Medical Center, Inc., Boston, MA (US)

(21) Appl. No.: 17/774,385

(22) PCT Filed: Nov. 5, 2020
(86) PCT No.: PCT/US2020/059152

§ 371 Date: May 4, 2022

Related U.S. Application Data
(60) Provisional application No. 62/931,094, filed on Nov. 5, 2019.

(51) Int. Cl. A61K 39/04; C12N 1/20; C12N 1/36; G01N 33/68; A61P 31/06

(52) U.S. Cl. CPC A61K 39/04; C12N 1/20; C12N 1/36; G01N 33/6869; A61P 31/06; G01N 2333/54; A61K 2039/522

(57) ABSTRACT

Disclosed herein are immunogenic compositions (e.g., vaccines) for use in the treatment of mycobacteria infections and biomarkers for monitoring of therapeutic responsiveness to the immunogenic compositions in a subject (e.g., a human). In a first aspect, the disclosure features a pharmaceutical composition containing between 1×10\hat{ }2 CPU and 1×10\hat{ }10 CPU of a Mycobacterium tuberculosis strain (Mtb) with one or more mutations that ablate or reduce expression of LprG and Rv1410 (ΔLprG Mtb) in a volume of between 0.05 mL and 3 mL.

### STATEMENT AS TO FEDERALLY FUNDED RESEARCH

This invention was made with government support under Grant Nos. AI128751 and AI124377 awarded by the National Institutes of Health. The government has certain rights in the invention.

### BACKGROUND OF THE INVENTION

Bacille Calmette-Guerin (BCG), an attenuated whole cell vaccine based on Mycobacterium bovis, is the only licensed vaccine against Mycobacterium tuberculosis (Mtb), but its efficacy is suboptimal and it fails to protect against pulmonary tuberculosis. BCG is currently the only approved TB vaccine for use in humans. While it protects against childhood TB meningitis, it provides only limited protection in adulthood (Trunz et al. Lancet. 367, 1173-1180 (2006)).

BCG, which is an attenuated strain of Mycobacterium bovis, the mycobacterial agent of bovine tuberculosis, has lost a number of virulence factors, such as ESAT6 and CFP-10 (Aguilo et al. Nature Communications. 8, 16085 (2017)). BCG vaccination is almost universal in high burden TB regions of the world. Therefore, any novel vaccination regimen will need to surpass BCG efficacy. Recent failures of other Mtb subunit vaccines suggest that the development of improved TB vaccines is warranted (Tameris et al. Lancet. 381, 1021-1028 (2013); Tameris et al. Lancet Respir Med. 7(9):757-770 (2019)).

### SUMMARY OF THE INVENTION

The present disclosure provides immunogenic compositions (e.g., vaccines) for use in the treatment of mycobacteria infections and biomarkers (e.g., any of the biomarkers described herein) for monitoring of therapeutic responsiveness to immunogenic compositions (e.g., vaccines), including those described herein.

In a first aspect, the disclosure features a pharmaceutical composition containing between 1×10² CFU and 1×10¹⁰ CFU of a Mycobacterium tuberculosis strain (Mtb) with one or more mutations that ablate or reduce expression of LprG and Rv1410 (ΔLprG Mtb) in a volume of between 0.05 mL and 3 mL.

In some embodiments, the ΔLprG Mtb is live or whole cell or is inactivated, such as by heat, fixation, or radiation.

In certain embodiments, the pharmaceutical composition further contains a pharmaceutically acceptable vehicle, diluent, and/or excipient.

In some aspects, the pharmaceutical composition contains an adjuvant.

In any of the preceding embodiments, the pharmaceutical composition is in a form suitable for subcutaneous, intradermal, intravenous, intramuscular, transdermal, parenteral, intranasal, respiratory, perioral, sublingual, oral, or topical administration.

In other embodiments, the composition is in lyophilized, solid, or liquid form.

In any of the preceding embodiments, the ΔLprG Mtb further contains one or more additional genetic modifications (e.g., deletions (e.g., deletions in coding regions (e.g., genes (e.g., virulence genes)), or in non-coding regions (e.g., regulatory sequences, CRISPR sequences, or mobile genetic elements)), substitutions (e.g., codon deoptimization), and/or insertions (e.g., insertion of antigenic sequences (e.g., antigenic Mtb peptides (e.g., Ag85B, ESAT-6, and/or TB10.4 peptides) or heterologous antigenic peptides (e.g., an antigenic peptide from a pathogen (e.g., bacterial, viral, parasitic, or fungal pathogen)))).

In any of the preceding embodiments, the ΔLprG Mtb further contains one or more mutations that ablate or reduce expression of one or more additional genes, such as, for example, a gene selected from the group consisting of fad26, phoP, sigH, pan, RD-1, LysA, and leu.

In certain aspects, the ΔLprG Mtb encodes one or more transgenes. In some embodiments, at least one transgene encodes a cytokine, a chemokine, an immunoregulatory agent, or a therapeutic agent. In other embodiments, at least one transgene contains a foreign antigen.

In some embodiments, the composition is capable of inducing an immune response in a human.

In other embodiments, the composition is a vaccine.

A second aspect features a method of inducing an immune response in a human including administering the pharmaceutical composition of the first aspect of the disclosure to the human.

In some embodiments of the second aspect of the disclosure, administering the pharmaceutical composition treats or prevents a disease, reduces symptoms of a disease, prevents the reemergence of a disease from latency, reduces sequela of a disease, and/or reduces the transmissibility of a disease. The disease may be an infectious disease. In some embodiments, the infectious disease is caused by one or more bacteria. In some embodiments, one or more bacteria are Mycobacterium spp. In certain embodiments, at least one Mycobacterium spp. is selected from M. tuberculosis, M. leprae, M. bovis, M. africanum, M. avium, M. canetti, M. chelonae, M. fortuitum, M. gordonae, M. hiberniae, M. intracellulare, M. kansasii, M. marinum, M. microti, M. paratuberculosis, M. phlei, M. pinnipedii, M. scrofulaceum, M. simiae, M. smegmatis, M. szulgai, M. ulcerans, M. vacca, and M. xenopi. The at least one Mycobacterium spp. may be M. tuberculosis.

The composition may be administered as a single dose or as a plurality of doses. In some embodiments, the doses are administered at least one day apart. The plurality of doses may be administered at least two weeks apart. In other embodiments, the composition is administered twice (e.g., as a prime and boost).

In other embodiments, the composition can be delivered by subcutaneous, intradermal, intravenous, intramuscular, transdermal, parenteral, intranasal, respiratory, perioral, sublingual, oral, or topical administration.

The composition can be administered as either a priming component or a boosting component in a prime-boost immunization. For example, the composition can be administered as the priming component and the boosting component can be selected from a whole cell vaccine (e.g., BCG, MTBVAC, VPM1002, or DAR-901), a recombinant vector vaccine (e.g., MVA85A, ChAdOx1.PPE15, TB/FLU-04L, Ad5Ag85A, or AERAS-402), or a subunit vaccine (e.g., M72, RUTI, H107, or CysVac2/Advax).

In some embodiments of the second aspect of the disclosure, the composition is administered as the boosting component and the priming component can be selected from a whole cell vaccine (e.g., BCG, MTBVAC, VPM1002, or DAR-901), a recombinant vector vaccine (e.g., MVA85A, ChAdOx1.PPE15, TB/FLU-04L, Ad5Ag85A, or AERAS-402), or a subunit vaccine (e.g., M72, RUTI, H107, or CysVac2/Advax).

In a third aspect, the disclosure features a method of monitoring responsiveness of a subject (e.g., a mammal, such as a human) to an immunogenic composition that has been administered for treatment or prevention of an infection by detecting a level of a biomarker (e.g., IL-17A, IL-6, IP-10, MIP-2, MIP-1α, MCP-1, IL-2, IL-10, IFNγ, TNFα, TNF-α-secreting CD4⁺ T cells, IFN-γ/TNF-α-secreting CD4⁺ T cells, or PD-1-negative Ag-specific CD4⁺ T cells) in a sample from the subject that is obtained after administration of the immunogenic composition, in which detection of the level of the biomarker in the sample that is higher than a reference level identifies the subject as responsive to the treatment and a level that is lower than or equal to the reference level identifies the subject as unresponsive to the treatment. In some embodiments, one or more additional biomarkers are detected. In some embodiments, the sample is a blood sample (e.g., a whole blood sample, a serum sample, or a plasma sample), a bronchoalveolar lavage sample, or a lung biopsy sample. In other embodiments, the reference level of the biomarker is a level present in the 5^{th} percentile of a reference population, or greater, such as in the 50^{th} percentile of a reference population or in the 95^{th} percentile of a reference population (e.g., in the 60^{th}, 70^{th}, 80^{th}, 85^{th}, 90^{th}, 95^{th}, or 99^{th} percentile or greater).

In a fourth aspect, the disclosure features a method of monitoring responsiveness of a subject (e.g., a mammal, such as a human) to an immunogenic composition that has been administered for treatment or prevention of an infection by detecting a level of IL-17A in a sample from the subject that is obtained after administration of the immunogenic composition, in which detection of the level of IL-17A in the sample that is higher than a reference level identifies the subject as responsive to the treatment and a level that is lower than or equal to the reference level identifies the subject as unresponsive to the treatment. The immunogenic composition can be a vaccine. In an embodiment, the reference level of IL-17A is the level of IL-17A present in a sample from the subject prior to administration of the immunogenic composition. In other embodiments, the reference level of IL-17A is a level of IL-17A present in the 5^{th} percentile of a reference population, or greater, such as in the 50^{th} percentile of a reference population or in the 95^{th} percentile of a reference population.

The level of IL-17A may be detected between 1 minute and 12 weeks after administration of the immunogenic composition to the subject.

In other embodiments, the infection is a bacterial infection, such as an infection by one or more Mycobacterium spp. (e.g., a Mycobacterium spp. selected from M. africanum, M. avium, M. bovis, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi, preferably, M. tuberculosis).

The immunogenic composition can be a M. tuberculosis immunogenic composition or vaccine. In some embodiments, the immunogenic composition or vaccine contains the composition of the first aspect of the disclosure alone or in combination with one or more of BCG, MTBVAC, VPM1002, DAR-901, MVA85A, ChAdOx1.PPE15, TB/FLU-04L, Ad5Ag85A, AERAS-402, M72, RUTI, H107, or CysVac2/Advax.

In an embodiment, the sample is blood; optionally serum or plasma.

In some embodiments, one or more additional biomarkers are detected. In some embodiments, the one or more additional biomarkers are IL-6, IP-10, MIP-2, MIP-1α, MCP-1, IL-2, IL-10, IFNγ, TNFα, TNF-α-secreting CD4⁺ T cells, IFN-γ/TNF-α-secreting CD4⁺ T cells, PD-1-negative Ag-specific CD4⁺ T cells, and/or PD-1-positive Ag-specific CD4⁺ T cells.

In a fifth aspect, the disclosure features a kit containing a composition of the first aspect of the disclosure and a reagent for measuring a level of IL-17A in a sample.

In some embodiments of the fifth aspect of the disclosure, the kit further contains one or more of BCG, MTBVAC, VPM1002, DAR-901, MVA85A, ChAdOx1.PPE15, TB/FLU-04L, Ad5Ag85A, AERAS-402, M72, RUTI, H107, or CysVac2/Advax.

In certain embodiments of the fifth aspect of the disclosure, the reagent is an immunoassay reagent. In some embodiments, the reagent is for use in an ELISA. In an embodiment, the sample is blood; optionally serum or plasma.

The kit may further contain instructions for use and/or one or more samples containing a known amount of IL-17A.

In some embodiments of the fifth aspect of the disclosure, the kit further contains one or more reagents for detecting one or more additional biomarkers (e.g., IL-6, IP-10, MIP-2, MIP-1α, MCP-1, IL-2, IL-10, IFNγ, TNFα, TNF-α-secreting CD4⁺ T cells, IFN-γ/TNF-α-secreting CD4⁺ T cells, PD-1-negative Ag-specific CD4⁺ T cells, and/or PD-1-positive Ag-specific CD4⁺ T cells).

### BRIEF DESCRIPTION OF THE DRAWINGS

The accompanying drawings are included to illustrate embodiments of the invention and further an understanding of its implementations.

The application file contains at least one drawing executed in color. Copies of this patent or patent application with color drawings will be provided by the Office upon request and payment of the necessary fee.

FIGS. 1A-1I show the protective efficacy of ΔLprG and BCG vaccines in C57BL/6J and Balb/cJ Mice and attenuation in SCID mice. C57BL/6J or Balb/cJ mice were vaccinated with 100 uL of O.D. 1.0 log-phase culture of either BCG Serum Statens Institute (SSI) or H37RvΔlprG-rv1410c (ΔLprG) subcutaneously in the left flank. Peripheral blood mononuclear cells (PBMC) were collected 2 weeks post-vaccination. Percent cytokine positive (% Cyt⁺) antigen-specific T lymphocyte responses are shown as measured by intracellular cytokine staining (ICS) in CD4⁺ CD44⁺ T lymphocytes following stimulation with purified protein derivative (PPD, Synbiotic Tuberculin OT). Mice were aerosol-challenged with 75 CFU of Mtb H37Rv. Colony-forming units (CFU) were counted from lung or spleen at week 4 following challenge; C57BL/6 (FIGS. 1A-1C) and Balb/cJ (FIGS. 1D-1F) mice. SCID mice received approximately 75 CFU of aerosolized Mtb H37Rv, BCG SSI, or ΔLprG. CFU from lung (FIG. 1G) or spleen (FIG. 1H) of SCID mice 4 weeks post-infection. (FIG. 1I) Survival curve from (FIGS. 1G, 1H) showing survival of mice receiving aerosolized ΔLprG and BCG vaccines out to 100 days. Kruskall-Wallis with Dunn's correction for multiple comparisons; * p<0.05; ** p<0.01; *** p<0.001; **** p<0.0001. Red bar=median. Data is one representative experiment out of three with 5-15 mice per group for C57BL/6J mice. Balb/cJ and SCID challenges were performed once with 5 mice per group.

FIGS. 2A-2H show the induction of pro-inflammatory cytokines by ΔLprG and BCG vaccines in C3HeB/FeJ mice. FIG. 2A is a schematic showing a vaccination regimen. C3HeB/FeJ mice were vaccinated with 100 uL of O.D. 1.0 log-phase culture of either BCG Serum Statens Institute (SSI) or H37RvΔlprG-rv1410c (ΔLprG) subcutaneously in the left flank. Serum for Luminex cytokine analysis was collected on days 1 and 7 after vaccination. Peripheral blood mononuclear cells (PBMC) were collected at weeks 2, 6, and 9 post-vaccination. FIG. 2B is a heatmap showing median log 2 fold-change of serum cytokine levels as compared to naïve mice in BCG and ΔLprG vaccinated mice on days 1 and 7. FIGS. 2C-2H are graphs showing individual cytokine levels in serum on days 1 and 7; bar represents median values;* p<0.05; ** p<0.01, Mann-Whitney U test (BCG vs. ΔLprG). LLOQ represents lower limit of quantification. Luminex assays were performed twice with 5-8 mice per group. Data is representative of individual experiments.

FIG. 3 is a set of graphs showing the effect of LprG-Rv1410 mutation on host recognition of T cell epitopes. C57BL/6J mice were injected subcutaneously with two doses of 100 uL OD600=1.0 stocks of either BCG, H37Rv::Δrv1411c-rv1410c, or H37Rv and splenocytes were harvested 9 days post-boost. Percent cytokine positive CD4⁺ and CD8⁺ CD44⁺ antigen-specific splenocytes as measured by intracellular cytokine staining (ICS) following stimulation with 15-mer overlapping peptide pools spanning the entire Ag85B, ESAT-6, and TB10.4 proteins. Percentages reflect subsets of cytokine secreting cell populations from Boolean analysis (FlowJo v10) of all possible cytokine combinations (IFNγ, TNF-α, IL-2, IL-17A, and IL-10). Data representative of a single experiment with 5-8 animals per group.

FIGS. 4A-4F are graphs showing the immunogenecity of BCG and ΔLprG vaccines in C3HeB/FeJ mice. PBMC were collected at week 2, 6, and 9 following vaccination (FIGS. 4A, 4B). Lung leukocytes (FIGS. 4C, 4D) and splenocytes (FIGS. 4E, 4F) were collected 2 weeks following vaccination. Percent cytokine positive (% Cyt⁺) antigen-specific T lymphocyte responses as measured by intracellular cytokine staining (ICS) in CD4⁺ (A,C,E) and CD8⁺ (FIGS. 4B, 4D, 4F) CD44⁺ T lymphocytes following stimulation of PBMC, lung leukocytes, or splenocytes with purified protein derivative (PPD, Synbiotic Tuberculin OT). Percentages reflect subsets of cytokine secreting cell populations from Boolean analysis (FlowJo v10) of all possible cytokine combinations (IFNγ, TNF-α, IL-2, IL-17A, and IL-10); * p<0.05; ** p<0.01, Kruskall-Wallis for total % Cyt+ cells with Dunn's corrections for multiple comparisons. Data is representative of individual experiments. PBMC ICS was performed 4 times with 5-10 animals per vaccine group. Lung leukocytes and splenocyte analyses were performed once with 5-10 animals per group. Data presented is from two different replicates.

FIGS. 5A-5M are graphs and histology images showing the protective efficacy of ΔLprG and BCG vaccines against Mtb challenge in C3HeB/FeJ mice. FIGS. 5A and 5B are graphs showing colony-forming units (CFU) from lung (FIG. 5A) or spleen (FIG. 5B) at week 4 following challenge with 75 CFU of aerosolized Mtb H37Rv. FIGS. 5C and 5D are graphs showing histopathology on lungs from naïve and vaccinated C3HeB/FeJ mice following aerosol Mtb challenge showing individual granuloma size (mm²) in lungs from mice at week 4 following challenge (FIG. 5C) and percent of granulomas that contain visible acid-fast organisms (FIG. 5D). Dots represent individual animals (FIG. 5C) and individual granuloma measurements (FIG. 5D). Kruskall-Wallis with Dunn's correction for multiple comparisons; * p<0.05; ** p<0.01; *** p<0.001; **** p<0.0001. Red bar=median. Representative images of Ziehl-Neelsen acid-fast staining in affected lung from Naïve mice (FIG. 5E), BCG vaccinated mice (FIG. 5F), and ΔLprG vaccinated mice (FIG. 5G) 4 weeks post-Mtb aerosol challenge, at 100× magnification. Representative images of Ziehl-Neelsen acid-fast staining in affected lung from Naïve mice (FIG. 5H) and ΔLprG vaccinated mice (FIG. 5I) 4 weeks post-Mtb challenge, at 400× magnification. Percent cytokine positive (% Cyt⁺) antigen-specific T lymphocyte responses as measured by intracellular cytokine staining (ICS) following stimulation with PPD (Synbiotic; Tuberculin OT) in CD4⁺ (FIG. 5J) or CD8⁺ (FIG. 5K) CD44⁺ T lymphocytes isolated from lungs of C3HeB/FeJ mice at week 4 following aerosol Mtb challenge; * p<0.05, Kruskall-Wallis with Dunn's corrections for multiple comparisons. Pearson's correlation (R²) of TNFα⁺ (FIG. 5L) and IFNγ⁺ TNFα⁺ (FIG. 5M) CD44⁺ CD4⁺ T cells in lung post-Mtb challenge with Log 10 CFU in lung following challenge. Protection data (FIGS. 5A-5D) is representative of 3 individual challenge experiments with 8-10 mice per group. Lung leukocyte levels post-challenge and CFU correlation analyses (FIGS. 5J-5M) are representative of 2 independent Mtb challenges with 8-10 mice per group. Red=ΔLprG-vaccinated; Blue=BCG-vaccinated; Black=Naïve mice.

FIG. 6 is a graph showing the dose titration and protective efficacy of BCG Pasteur and ΔLprG vaccines in C57BL/6J mice. Female 6-8 wk old C57BL/6J mice were immunized subcutaneously with either 1×10⁶ or 1×10⁷ of freshly propagated vaccine cultures 8 weeks prior to aerosol challenge with 75 CFU of H37Rv Mtb. Lungs were homogenized and CFU enumerated 4 weeks post-challenge after growth on Middlebrook 7H10 agar. Data represents a single experiment performed once with 5 mice per group.

FIGS. 7A-7B are graphs showing cytokine secretion in splenocytes from naïve and vaccinated mice following Mtb challenge. Naïve, BCG, or ΔLprG vaccinated mice were challenged with 75 CFU Mtb H37Rv. Splenocytes were collected and stimulated with PPD. Percent cytokine secreting CD4⁺ (FIG. 7A) and CD8⁺ (FIG. 7B) CD44⁺ T cells are shown. Data representative of one of two experimental replicates with 5 mice per group.

FIGS. 8A-8E are graphs showing the cellular immune responses in C3HeB/FeJ lung post-Mtb challenge in BCG and ΔLprG vaccinated mice. Percent (FIG. 8A) PD-1-negative (PD-1⁻) and (FIG. 8B) PD-1-positive (PD-1⁺) cytokine-positive (Cyt⁺) CD4⁺ T cells in lung as measured by intracellular cytokine-staining after stimulation with PPD (Synbiotic; Tuberculin OT) from Naïve and vaccinated mice 4 weeks post-Mtb challenge. (FIG. 8C) Percent PD-1⁺ CD44⁺ CD4⁺ cytokine-negative T lymphocytes. Kruskall-Wallis with Dunn's corrections for multiple comparisons; * p<0.05; ** p<0.01; *** p<0.001. Red bar=Median. (FIG. 8D) Pearson's correlation (R²) of percent PD-1⁺ cytokine-negative CD44⁺ CD4⁺ T lymphocytes with CFU in lung following challenge. Pearson's correlation of PD-1− Cyt⁺ CD44⁺ CD4⁺ T lymphocytes (FIG. 8E, left) and PD-1⁺ CD44⁺ CD4⁺ T lymphocytes (FIG. 8E, right) with CFU in lung following challenge. (FIGS. 8D-8E) Red=ΔLprG-vaccinated; Blue=BCG-vaccinated; Black=Naïve mice. Data representative of 2 independent experiments with 5-8 mice per group.

FIGS. 9A-9D are graphs showing the PD-1-negative CD4⁺ T cell responses in naïve and vaccinated C3HeB/FeJ mice following Mtb challenge. Naïve, BCG, or ΔLprG vaccinated mice were challenged with 75 CFU Mtb H37Rv. T cells from lung were collected and stimulated with PPD. PD-1-negative populations shown in each panel. % IFN-γ, TNF-α, IL-2 positive PD-1⁻ CD4⁺ T cells are shown (FIGS. 9A-9D). Kruskall-Wallis with Dunn's corrections for multiple comparisons;* p<0.05; ** p<0.01; *** p<0.001. Red bar indicates median values. Data representative of 2 experimental replicates with 5-8 mice per group.

FIGS. 10A-10C are graphs showing the induction of lung Th17 cells and serum IL-17 by ΔLprG and BCG vaccines. (FIG. 10A) Percent cytokine positive (Cyt+) Ag-specific IL-17A⁺ CD44⁺ CD4⁺ T cells in lung at week 4 after Mtb challenge as measured by intracellular cytokine staining (ICS) post-stimulation with PPD (Synbiotic; Tuberculin OT). (FIG. 10B) Percent total IL17A⁺ PD-1⁻ CD44⁺ CD4⁺ T lymphocytes in lung at week 4 following challenge. Kruskall-Wallis with Dunn's corrections for multiple comparisons; * p<0.05. Red bar=median. (FIG. 10C) Serum IL-17 at week 2 after vaccination via Luminex. Kruskall-Wallis with Dunn's corrections for multiple comparisons;* p<0.05; ** p<0.01. Red bar=median. LLOQ=Lower Limit of Quantification. Lung leukocyte Th17 data and Luminex data representative of 2 independent experiments with 5-8 mice per group.

FIGS. 11A-11F are graphs and a heat map showing the correlations of serum cytokine levels following vaccination with CFU in lung following Mtb challenge. Serum cytokine levels from naïve and vaccinated mice were assessed at week 2 after vaccination by Luminex assays. Mice were challenged with 75 CFU Mtb. Pearson's correlations of cytokine levels with CFU in lung following challenge for (FIG. 11A) IL-17, (FIG. 11B) IL-6, (FIG. 11C) IP-10, (FIG. 11D) MIP1-α, and (FIG. 11E) MIP-2. Red=ΔLprG vaccinated; Blue=BCG vaccinated; Black=Naïve mice. (FIG. 11F) Heatmap of the normalized cytokines serum levels using z-score approach, correlated with Log 10 CFU in lung (purple) and spleen (green) two weeks post-vaccination in Naïve (coral), BCG (light blue), and ΔLprG (green) vaccinated mice. CFU levels were used as a continuous variable. Each column represents an individual animal and each row represents an individual cytokine. Z-score levels range from blue (negatively correlated with CFU) to red (positively correlated with CFU). Serum cytokine correlation analyses are representative of two experiments with 8-10 mice per group.

FIGS. 12A-12E are a heatmap graphs showing the serum cytokine secretion in naïve and vaccinated mice at week 2 following vaccination. (FIG. 12A) Heatmap of median log 2 fold-change cytokine and chemokine secretion in sera from BCG and ΔLprG vaccinated mice as compared to naïve animals at week 2 following vaccination as measured by Luminex assays. (FIGS. 12B-12E) Graphs showing serum cytokine levels from naïve and vaccinated mice. Bars represent median values. LLOQ represents lower limit of quantification. Data representative of two experimental replicates with 5 mice per group.

FIGS. 13A-13E are graphs showing the validation of IL-17 serum immune correlate using high-sensitivity Luminex and Mtb Erdman challenge. Serum was collected from mice at week 2 following vaccination with BCG or ΔLprG as well as from naïve mice. (FIG. 13A) Serum IL-17A levels were evaluated using a high-sensitivity Luminex assay. vKruskall-Wallis with Dunn's corrections for multiple comparisons; **p<0.01. vLower limit of quantification=0.976 pg/mL. Red lines reflect median values. Log CFU in lung (FIG. 13B) and spleen (FIG. 13C) in C3HeB/FeJ in unvaccinated (Naïve) mice and in BCG or ΔLprG vaccinated mice at week 4 following aerosol challenge with 75 CFU Mtb Erdman. Pearson's correlation of lung (FIG. 13D) and spleen (FIG. 13E) Log CFU following challenge with serum IL-17A levels following vaccination. Red=ΔLprG vaccinated; Blue=BCG vaccinated; Black=Naïve mice. Validation of serum IL-17 correlation with CFU following Erdman challenge was performed once with 4-8 mice per group.

FIG. 14 is a graph showing the receiver operator curve for high-sensitivity IL-17A serum Luminex assay. Mice were vaccinated with BCG and ΔLprG as described and sera collected for Luminex. The x-axis represents baseline percentiles and the y-axis is the probability that the BCG vaccinated values (filled squares) or ΔLprG vaccinated values (filled circles) were greater than or equal to the baseline percentile threshold as calculated using Graphpad prism v8. Data presents a single experiment performed once with 4-8 mice per group.

FIG. 15 is a set of graphs showing the serum IL-6 levels in vaccinated mice challenged with Mtb Erdman. IL-6 cytokine levels in sera from BCG and ΔLprG vaccinated mice as compared to naïve animals at week 2 following vaccination as measured by Luminex assays. Correlations of serum IL-6 levels lung and spleen CFU in mice challenged with Mtb Erdman four weeks post-challenge. Mtb Erdman challenge was performed once with 4-8 mice per group.

### DETAILED DESCRIPTION OF THE INVENTION

### I. Introduction

The present disclosure provides therapeutic and diagnostic methods and compositions for treating and/or preventing infectious disease (e.g., treating and/or reducing the likelihood of developing an infection, such as an Mtb infection) using, e.g., an immunogenic composition (e.g., a vaccine) containing ΔLprG Mtb. The invention is based, at least in part, on the discovery that an immunogenic bacterial composition (e.g., a vaccine, such as a ΔLprG Mtb-containing composition) that induces a strong Th17 response can induce a robust immunological response (e.g., production of antigen-specific cytokine-secreting CD4⁺ T cells) and can provide improved protective efficacy over compositions which induce weaker Th17 responses. The invention is also based, at least in part, on the discovery that biomarkers that indicate a strong Th17 response (e.g., IL-17A) can be used to monitor the responsiveness of a subject to a vaccine, such as the ΔLprG Mtb vaccine or immunogenic composition disclosed herein.

### II. Definitions

As used herein, the term “about” means ±10% of the recited value.

An “antigen” refers to any agent, generally a macromolecule, which can elicit an immunological response in an individual. As used herein, “antigen” is generally used to refer to a polypeptide molecule or portion thereof which contains one or more epitopes. Furthermore, for the purposes of the present disclosure, an “antigen” also includes a polypeptide having modifications, such as deletions, additions, and substitutions (generally conservative in nature) to the native sequence, so long as the polypeptide maintains sufficient immunogenicity. These modifications may be deliberate, for example through site-directed mutagenesis, or may be accidental, such as through mutations of hosts which produce the antigens.

As used herein, the term “epitope” generally refers to a site on a target antigen that is recognized by an immune receptor, such as a T-cell receptor and/or an antibody. An epitope may be a contiguous epitope, where the site recognized is a conformation of contiguous amino acid residues of a polypeptide, or a discontiguous epitope, where the site recognized is a portion of a folded polypeptide where the amino acid residues interacting with the immune receptor are not consecutive amino acids. The epitope may also include glycopeptides and carbohydrate epitopes. A single antigenic molecule may include several different epitopes.

The term “immunogenic composition” as used herein, is defined as material used to provoke an immune response and may confer immunity after administration of the immunogenic composition to a subject.

The term “vaccine” as used herein, is defined as material used to provoke an immune response and that confers immunity for a period of time after administration of the vaccine to a subject.

By “pharmaceutically acceptable diluent, excipient, carrier, or adjuvant” is meant a diluent, excipient, carrier, or adjuvant that is physiologically acceptable to the subject while retaining the therapeutic properties of the pharmaceutical composition with which it is administered. One exemplary pharmaceutically acceptable carrier is physiological saline. Other physiologically acceptable diluents, excipients, carriers, or adjuvants and their formulations are known to one skilled in the art (see, e.g., U.S. Pub. No. 2012/0076812).

The term “adjuvant” refers to a pharmacological or immunological agent that modifies the effect of other agents (e.g., vaccines) while having few if any direct effects when given by itself. They are often included in vaccines to enhance the recipient's immune response to a supplied antigen while keeping the injected foreign material at a minimum.

An “immune response” against an antigen of interest is the development in a mammalian subject (e.g., a human) of a humoral and/or a cellular immune response to that antigen. For purposes of the present disclosure, a “humoral immune response” refers to an immune response mediated by antibody molecules, a “cellular immune response” is one mediated by T-lymphocytes and/or other white blood cells. In addition, “sufficient immunogenicity,” as referred to herein, means a magnitude of an immune response that is sufficient to treat or prevent disease (e.g., Mtb infection), to alleviate one or more symptoms of a disease (e.g., Mtb infection), and/or to reduce a period of time during which a subject is suffering from a disease (e.g., Mtb infection). A mammalian subject to be administered a ΔLprG Mtb composition disclosed herein may be any member of the subphylum cordata, including, without limitation, humans and other primates, including non-human primates, such as chimpanzees and other apes and monkey species; farm animals, such as cattle, sheep, pigs, goats, and horses; domestic mammals, such as dogs and cats; laboratory animals including rodents, such as mice, rats and guinea pigs; birds, including domestic, wild, and game birds, such as chickens, turkeys and other gallinaceous birds, ducks, geese, and the like. The terms do not denote a particular age. Thus, both adult and newborn individuals are intended to be covered. The methods described herein can be intended for use in any of the above vertebrate species, since the immune systems of all of these vertebrates operate similarly. The subject is preferably a human.

As used herein, the term “prophylactically or therapeutically effective dose” means a dose in an amount sufficient to elicit an immune response to one or more epitopes of a polypeptide incorporated into a vector of the disclosure and/or to alleviate, reduce, cure or at least partially arrest symptoms and/or complications from a disease or infection for which the vector is administered.

As used herein, the term “treatment,” in the context of treating subjects with or at risk of developing and/or transmitting disease, means an action taken that can eliminate, reduce, alleviate one or more symptoms or signs of disease; prevent, delay, or reduce a course of disease; prevent or reduce the likelihood of disease transmission; and/or prevent, eliminate, reduce, alleviate, or delay sequelae. Preferably, the action taken includes the compositions or methods described herein either alone or in combination with other known compositions or methods.

As used herein, by “administering” is meant a method of giving a dosage of a composition (e.g., a pharmaceutical composition (e.g., an immunogenic composition (e.g., a vaccine))) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in cremes, or in lipid compositions. The method of administration can vary depending on various factors (e.g., the components of the composition being administered and the severity of the condition being treated).

The term “biomarker” as used herein refers to an indicator, e.g., predictive, diagnostic, and/or prognostic, which can be detected in a sample (e.g., a blood sample (e.g., a whole blood sample, a serum sample, or a plasma sample), a bronchoalveolar lavage sample, or a biopsy sample (e.g., a lung biopsy sample)), such as a sample from a subject. The biomarker may serve as an indicator of a treatment response (e.g., a treatment response to an immunogenic composition of ΔLprG Mtb (e.g., a vaccine), other vaccines (e.g., BCG, MTBVAC, VPM1002, DAR-901, MVA85A, ChAdOx1.PPE15, TB/FLU-04L, Ad5Ag85A, AERAS-402, M72 (e.g., M72/AS01_{E}), RUTI, H107, or CysVac2/Advax), or other immunogenic compositions). In some embodiments, a biomarker is a gene product (e.g., an RNA or a protein encoded by the gene). Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers. In some embodiments, one or more biomarkers may be IL-17A, IL-6, IP-10, MIP-2, MIP-1α, MCP-1, IL-2, IL-10, IFNγ, TNFα, TNF-α-secreting CD4⁺ T cells, IFN-γ/TNF-α-secreting CD4⁺ T cells, PD-1-negative Ag-specific CD4⁺ T cells, and/or PD-1-positive Ag-specific CD4⁺ T cells. In some embodiments, the biomarker is a cytokine. In some embodiments, the biomarker is a cell (e.g., an immune cell, e.g., a T cell, e.g., a CD4⁺ T cell, e.g., a Th17 cell). In some embodiments, the biomarker is an amount or level of IL-17A (e.g., an amount or level of IL-17A protein or mRNA). In some embodiments, the biomarker is an amount or level of IL-17A protein.

As used herein, the term “sample” is a composition that is obtained or derived from a subject that contains a cellular and/or other molecular entity that is to be characterized and/or identified, for example based on physical, biochemical, chemical and/or physiological characteristics. A sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid. The sample may also be primary or cultured cells or cell lines. The sample may contain compounds which are not naturally intermixed with the tissue in nature such as preservatives, anticoagulants, buffers, fixatives, wax, nutrients, antibiotics, or the like.

A “subject” is a vertebrate, such as a mammal (e.g., a primate and a human, in particular a human with underlying health conditions (e.g., hypertension, diabetes, or cardiovascular disease)). Mammals also include, but are not limited to, farm animals (such as cows), sport animals (e.g., horses), pets (such as cats, and dogs), mice, rats, bats, civets, and raccoon dogs. A subject to be treated according to the methods described herein (e.g., a subject in need of protection from an Mtb infection or having an Mtb infection may be one who has been diagnosed by a medical practitioner as having such a need or infection. Diagnosis may be performed by any suitable means. A subject in whom the development of an infection is being prevented may or may not have received such a diagnosis. One skilled in the art will understand that a subject to be treated according to the present invention may have been subjected to standard tests or may have been identified, without examination, as one with a suspected infection or at high risk of infection due to the presence of one or more risk factors (e.g., exposure to Mtb, for example, due to travel to an area where Mtb infection is prevalent).

### III. Compositions and Methods

The disclosure is based, in part, on the recognition that a mycobacteria strain, the ΔLprG mycobacterium, can be administered as an immunogenic composition (e.g., a vaccine) and can be used to treat or prevent (e.g., eliminate or reduce the likelihood of developing) a mycobacterial infection or to reduce the term of, or one or more symptoms of, a mycobacterial infection. Such vaccines are useful, for example, for treating a subject, such as a human, having, or at risk of developing, an infection by mycobacteria.

### The ΔLprG Mtb Strain

The disclosure provides compositions, such as immunogenic compositions (e.g., vaccines), that include ΔLprG mycobacteria (Mtb).

Mtb lacking the virulence genes lprG and rv1410c (designated ΔLprG) is highly attenuated in immune deficient mice. ΔLprG Mtb have stable genetic, unmarked deletions in two genes, which render the Mtb less pathogenic than BCG in immune deficient mice. The genes rv1411c-rv1410c encode a lipoprotein (LprG) and transmembrane efflux pump (Rv1410) that have been shown to be conditionally essential for in vivo survival in mice (Farrow and Rubin. J Bacteriol. 190, 1783-1791 (2008) and Martinot et al. PLoS Pathog. 12, e1005351 (2016)). The operon containing these genes is involved in lipid transport (Farrow and Rubin. J Bacteriol. 190, 1783-1791 (2008)); disruption of the operon alters the lipid content of the Mtb cell wall and its metabolic state, which leads to marked attenuation in both immunocompetent (C57BL/6) and immunodeficient (Rag^{-/-}, SCID, phox^{-/-}, and infr^{-/-}) mice (Martinot et al. PLoS Pathog. 12, e1005351 (2016)).

The LprG lipoprotein is a potent TLR2 agonist (Drage et al. Nat Struct Mol Biol. 17, 1088-1095 (2010)) and has been hypothesized to play a role in host immune evasion by decreasing antigen presentation by macrophages in vitro (Harding and Boom. Nat Rev Micro. 8, 296-307 (2010)). LprG also binds immunomodulatory lipids that can prevent phagolysosome fusion, which may impact downstream MHC class II antigen processing (Shukla et al. PLoS Pathog. 10, e1004471 (2014) and Gaur et al. PLoS Pathog. 10, e1004376 (2014)). We discovered that deletion of the LprG-Rv1410 locus in Mtb resulted in an improved whole cell Mtb vaccine owing to the profound attenuation of Mtb observed with loss of the LprG-Rv1410 operon.

In some embodiments, ΔLprG Mtb can contain additional (e.g., 1, 2, 3, 4, 5, or more) genetic modifications. In some embodiments, additional genetic modifications can be deletions (e.g., deletions in coding regions (e.g., genes (e.g., virulence genes)), or in non-coding regions (e.g., regulatory sequences, CRISPR sequences, or mobile genetic elements)), substitutions (e.g., codon deoptimization), and/or insertions (e.g., insertion of antigenic sequences (e.g., antigenic Mtb peptides (e.g., Ag85B, ESAT-6, and/or TB10.4 peptides) or heterologous antigenic peptides (e.g., an antigenic peptide from a pathogen (e.g., bacterial, viral, parasitic, or fungal pathogen))). In some embodiments, an additional genetic modification is a deletion. In some embodiments, one or more deletions may be selected from the group containing fad26, phoP, sigH, pan, RD-1, LysA, and leu. Other genes that could be deleted in ΔLprG Mtb can include one or more of the genes or operons disclosed in, e.g., PCT publication no. PCT/US2012/032164, incorporated herein by reference. In some embodiments, an additional genetic modification is an insertion of one or more antigenic sequences (e.g., a plasmid containing one or more homologous and/or heterologous antigenic peptides).

### Formulation and Administration of Pharmaceutical Compositions

Formulation

The ΔLprG Mtb cells may be prepared for administration to a host (e.g., a human) by combining cells (e.g., live (e.g., whole cell) or heat-inactivated) with a pharmaceutically acceptable carrier to form a pharmaceutical composition.

Therapeutic formulations of compositions of the disclosure can be prepared using standard methods known in the art, such as by mixing the active component (e.g., the ΔLprG Mtb) with optional physiologically acceptable carriers, excipients or stabilizers (Remington's Pharmaceutical Sciences (20th edition), ed. A. Gennaro, 2000, Lippincott, Williams & Wilkins, Philadelphia, Pa.). Acceptable carriers include saline and/or buffers, such as phosphate, citrate and other organic acids. Other components can include, e.g., antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG. The carrier can be sufficiently pure to be administered therapeutically to a human subject. Those of relevant skill in the art are well able to prepare suitable solutions using, e.g., isotonic vehicles such as Sodium Chloride Injection, Ringer's Injection, or Lactated Ringer's Injection.

Optionally, the formulations of the disclosure can contain a pharmaceutically acceptable preservative. In some embodiments the preservative concentration can range from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the disclosure can include a pharmaceutically acceptable surfactant at a concentration of 0.005 to 0.02%. Stabilizers, buffers, antioxidants and/or other additives may be included, as required.

The ΔLprG Mtb may be incorporated into microparticles or microcapsules to prolong the exposure of the antigenic material to the subject animal and hence protect the animal against infection for long periods of time. The microparticles and capsules may be formed from a variety of well-known inert, biocompatible matrix materials using techniques conventional in the art. Suitable matrix materials include, e.g., natural or synthetic polymers such as alginates, poly(lactic acid), poly(lactic/glycolic acid), poly(caprolactone), polycarbonates, polyamides, polyanhydrides, polyortho esters, polyacetals, polycyanoacrylates, polyurethanes, ethytlenevinyl acetate copolymers, polystyrenes, polyvinyl chloride, polyvinyl fluoride, poly(vinyl imidazole), chlorosulphonated polyolefins, polyethylene oxide, and particularly agar and polyacrylates. The ΔLprG Mtb may be contained in small particles suspended in the water or saline.

Immunogenicity of the composition of the disclosure may be significantly improved if it is co-administered with an immunostimulatory agent, adjuvant, antibacterial agent, or other pharmaceutically active agent as are conventional in the art. Adjuvants may include but are not limited to salts, emulsions (including oil/water compositions), saponins, liposomal formulations, virus particles, polypeptides, pathogen-associated molecular patterns (PAMPS), nucleic acid-based compounds or other formulations utilizing certain antigens. Suitable adjuvants include, e.g., aluminum phosphate, aluminum hydroxide, OS21, Quil A (and derivatives and components thereof), calcium phosphate, calcium hydroxide, zinc hydroxide, glycolipid analogs, vegetable oils, alum, Freund's incomplete adjuvant, or Freund's incomplete adjuvant, octodecyl esters of an amino acid, muramyl dipeptides, polyphosphazene, lipoproteins, DC-Chol, DDA, cytokines, and other adjuvants and derivatives thereof. Other adjuvants include agents such as immunestimulating complexes (ISCOMs), synthetic polymers of sugars (CARBOPOL®), aggregation of the protein in the vaccine by heat treatment, aggregation by reactivating with pepsin treated (Fab) antibodies to albumin, endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

The ΔLprG Mtb may be contained in a mucosal bacterial toxin adjuvant such as the Escherichia co/i labile toxi (LT) and cholera toxin (CT) or in CpG oligodeoxynucleotide (CpG ODN)4 1. Another possible mucosal adjuvant Monophosphoryl lipid A (MPL), a derivative and less toxic form of LPS, when combined with liposomes was found to induce mucosal immunoprotective responses. The vaccine may optionally include additional immune modulating substances such as cytokines or synthetic IFN-g inducers such as poly I:C alone or in combination with the above-mentioned adjuvants. Still other adjuvants include microparticles or beads of biocompatible matrix materials. The microparticles may be composed of any biocompatible matrix materials as are conventional in the art, including but not limited to, agar and polyacrylates. The practitioner skilled in the art will recognize that other carriers or adjuvants may be used as well. For example, chitosan or any bioadhesive delivery system which may be used.

Pharmaceutical compositions according to the disclosure described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED50)).

Many strategies can be pursued to obtain controlled or extended release. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The pharmaceutical composition containing ΔLprG Mtb can be formulated, e.g., for administration subcutaneously, intranasally, intrapulmonarally, intramuscularly, intravenously, intradermally, percutaneously, intraarterially, intraperitoneally, intralesionally, intracranially, intraarticularly, intraprostatically, intrapleurally, intratracheally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in cremes, or in lipid compositions. The preferred method of administration can vary depending on various factors (e.g., the components of the composition being administered and the severity of the condition being treated). Formulations suitable for oral or nasal administration may consist of liquid solutions, such as an effective amount of the composition dissolved in a diluent (e.g., water, saline, or PEG-400), capsules, sachets, tablets, or gels, each containing a predetermined amount of the chimeric Ad5 vector composition of the disclosure. The pharmaceutical composition may also be an aerosol formulation for inhalation, for example, to the bronchial passageways. Aerosol formulations may be mixed with pressurized, pharmaceutically acceptable propellants (e.g., dichlorodifluoromethane, propane, or nitrogen). In particular, administration by inhalation can be accomplished by using, e.g., an aerosol containing sorbitan trioleate or oleic acid, for example, together with trichlorofluoromethane, dichlorofluoromethane, dichlorotetrafluoroethane, or any other biologically compatible propellant gas. The pharmaceutical composition containing ΔLprG Mtb is preferably formulated for subcutaneous, intranasal, intramuscular, intravenous, or intrapulmonary delivery using methods known in the art. The formulation of ΔLprG Mtb combined with the adjuvant is preferably selected to minimize side effects, such as inflammation, associated with vaccination or may improve the formulation's stability. The adjuvant may also have a role as an immunostimulant or as a depot.

ΔLprG Mtb may be delivered by the refinement of a nebulizer or via compact portable devices, such as the metered-dose inhaler (MDI) and the dry powder inhaler (DPI). Intransal delivery can occur via the nasal spray, dropper or nasal metered drug delivery device. ΔLprG Mtb may be delivered via a metered dose inhaler. Typically, only 10-20% of the emitted dose is deposited in the lung. The high velocity and large particle size of the spray causes approximately 50-80% of the drug aerosol to impact in the oropharyngeal region.

ΔLprG Mtb may be contained in a dry powder formulation such as but not limited to a sugar carrier system. The Sugar Carrier System could include lactose, mannitol, and/or glucose. Lactose, mannitol, and glucose are all approved by the FDA as carriers. There are also larger sugar particles such as lactose monohydrate, typically 50-100 micrometers in diameter, which remain in the naso-oropharynx but allows ΔLprG Mtb to travel through the respiratory tree into the alveoli.

If desired, ΔLprG Mtb may be contained in a liposomal formulation. Liposomes, like other inhaled particles reaching the alveoli, are cleared by macrophages. The processing, uptake and recycling of liposomal phospholipids occurs through the same mechanism as endogenous surfactant via the alveolar type II cells.

Compositions may be sterilized by conventional sterilization techniques or may be sterile filtered. The resulting aqueous solutions may be packaged for use as is, or lyophilized, the lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the ΔLprG Mtb and, if desired, one or more immunomodulatory agents, such as in a sealed package of tablets or capsules, or in a suitable dry powder inhaler (DPI) capable of administering one or more doses.

Administration

Pharmaceutical compositions (e.g., immunogenic compositions (e.g., vaccines)) of the disclosure can be administered to a subject (e.g., a human), pre- or post-exposure to an infective agent (e.g., bacteria, viruses, parasites, fungi) or pre- or post-diagnosis of a disease of a disease without an etiology traceable to an infective agent (e.g., cancer), to treat, prevent, ameliorate, inhibit the progression of, or reduce the severity of one or more symptoms of the disease in the subject. For example, compositions of the disclosure can be administered to a subject to treat tuberculosis. Examples of symptoms of diseases caused by a bacterial infection, such as tuberculosis, that can be treated using compositions of the disclosure include, for example, fever, muscle aches, coughing, sneezing, runny nose, sore throat, headache, chills, diarrhea, vomiting, rash, weakness, dizziness, bleeding under the skin, in internal organs, or from body orifices like the mouth, eyes, or ears, shock, nervous system malfunction, delirium, seizures, renal (kidney) failure, personality changes, neck stiffness, dehydration, seizures, lethargy, paralysis of the limbs, confusion, back pain, loss of sensation, impaired bladder and bowel function, and sleepiness that can progress into coma or death. These symptoms, and their resolution during treatment, may be measured by, for example, a physician during a physical examination or by other tests and methods known in the art.

Compositions of the disclosure may be administered to provide pre-exposure prophylaxis, post-exposure prophylaxis, after a subject has been exposed to a disease with a known etiology (e.g., Mtb infection) or an infective agent, such as a bacterium (e.g., Mtb), virus, parasite, or fungus, or after a subject has been diagnosed with a disease without an etiology traceable to an infective agent (e.g., cancer). The composition may be administered, for example, 1, 2, 3, 4, 5, 6, 7, 8, 9, 10, 15, 20, 30, 35, 40, 45, 50, 55, or 60 minutes, 2, 4, 6, 10, 15, or 24 hours, 2, 3, 5, or 7 days, 2, 4, 6 or 8 weeks, or even 3, 4, or 6 months pre-exposure or pre-diagnosis, or may be administered to the subject 1-30 minutes or 1, 2, 3, 4, 5, 6, 7, 8, 9, 10, 12, 20, 24, 48, or 72 hours, 3, 5, or 7 days, 2, 4, 6 or 8 weeks, 3, 4, 6, or 9 months, 1, 2, 3, 4, 5, 6, 7, 8, 9, 10, 15, or 20 years or longer post-diagnosis or post-exposure to the infective agent.

When treating disease (e.g., tuberculosis), compositions of the disclosure may be administered to the subject either before the occurrence of symptoms or a definitive diagnosis or after diagnosis or symptoms become evident. For example, a composition may be administered, e.g., immediately after diagnosis or the clinical recognition of symptoms or 2, 4, 6, 10, 15, or 24 hours, 2, 3, 5, or 7 days, 2, 4, 6 or 8 weeks, or even 3, 4, or 6 months after diagnosis or detection of symptoms.

A skilled person in the field familiar with the protocols, formulations, dosages and clinical practice associated with, e.g., the administration of an immunogenic composition, such as a vaccine (e.g., MTBVAC or BCG) can readily adapt known administration protocols for use with a pharmaceutical composition of the present disclosure. The immunogenic composition (e.g., a vaccine) can be administered in a manner compatible with the dosage and/or formulation, and in such amount as will be therapeutically effective and immunogenic. The quantity to be administered may depend on the subject to be treated (e.g., the age, body weight, the capacity of the subject's immune system to mount an immune response, the degree of protection desired, and general health of the subject being treated), the form of administration (e.g., as a solid or liquid), the manner of administration (e.g., by injection, inhalation, dry powder propellant), and the cells targeted (e.g., epithelial cells, such as blood vessel epithelial cells, nasal epithelial cells, or pulmonary epithelial cells).

In addition, single or multiple administrations of a composition of the present disclosure may be given (pre- or post-exposure and/or pre- or post-diagnosis) to a subject (e.g., one administration or administration two or more times). For example, subjects who are particularly susceptible to, for example, bacterial infection may require multiple treatments to establish and/or maintain protection against the virus. The magnitude of an immune response provided by a pharmaceutical composition described herein can be monitored by, for example, measuring serum cytokine levels (e.g., IL-17A) or measuring amounts of neutralizing secretory and serum antibodies. An increase in, for example, cytokine levels (e.g., IL-17A) or neutralizing antibodies is indicative of an immune response. The dosages may then be adjusted or repeated as necessary to trigger a desired level of immune response.

For example, an immune response triggered by a single administration (prime) of a composition of the disclosure may not be sufficiently potent and/or persistent to provide effective protection. Accordingly, in some embodiments, repeated administration (boost), such that a prime boost regimen is established, can significantly enhance humeral and cellular responses to the antigens of the composition.

The dose of a composition of the disclosure (e.g., the number ΔLprG Mtb CFU) or the number of treatments using a composition of the disclosure may be increased or decreased based on the severity of, occurrence of, or progression of, the disease in the subject (e.g., based on the severity of one or more symptoms of, e.g., a bacterial (e.g., Mtb) infection). A pharmaceutical composition of the disclosure can be administered in a therapeutically effective amount that provides an immunogenic and/or protective effect against an infective agent (e.g., Mtb) or target protein for a disease caused by a non-infective agent. For example, the subject can be administered at least about 1×10² CFU or between about 1×10² CFU and about 1×10¹⁰ CFU (e.g., 1×10², 1.1×10², 2×10², 5×10², 1×10³ 1×10⁴, 1×10⁵, 1×10⁶, 1×10⁷, 1×10⁸, 1×10⁹, 1×10¹⁰ CFU) of ΔLprG.

Suitable regimens for initial administration and booster shots are also variable but are typified by an initial administration followed by subsequent inoculations or other administrations. Thus, an immunogenic composition (e.g., a vaccine) of the present disclosure may be administered in a single dose or in a plurality of doses (e.g., 2, 3, 4, 5, or more doses) administered concurrently or about 1-30 minutes or about 1, 2, 3, 4, 5, 6, 7, 8, 9, 10, 12, 20, 24, 48, or 72 hours, or about 3, 5, or 7 days, or about 2, 4, 6 or 8 weeks, or about 3, 4, 6, or 9 months, 1, 2, 3, 4, 5, 6, 7, 8, 9, 10, 15, or 20 years or longer. In one embodiment, an immunogenic composition (e.g., a vaccine) of the present disclosure may be administered in two doses about 1-12 months apart. The subject may be vaccinated at any time, although it may be preferred to administer an immunogenic composition (e.g., a vaccine) of the present disclosure shortly (optimally about 10 days to two weeks) before anticipated exposure to an infected individual.

A composition may be administered alone or in combination with other treatments (e.g., vaccines, such as BCG, MTBVAC, VPM1002, DAR-901, MVA85A, ChAdOx1.PPE15, TB/FLU-04L, Ad5Ag85A, AERAS-402, M72 (e.g., M72/AS01_{E} (GSK)), RUTI, H107, or CysVac2/Advax), either simultaneously or sequentially (e.g., as a prime-boost) dependent upon the condition to be treated. The composition can be administered after vaccination with, e.g., BCG or another vaccine, and therefore may act as a boosting tuberculosis vaccine. Moreover, an immunogenic composition (e.g., a vaccine) of the present disclosure may be given after an initial subcutaneous inoculation followed by an intranasal or mucosal boost or vice versa.

### Methods of Prophylaxis or Treatment Using a Composition of the Disclosure

A pharmaceutical composition of the disclosure can be used as an immunogenic composition (e.g., a vaccine) for treatment and/or prophylaxis of a subject (e.g., a human) with a disease (e.g., cancer or a disease caused by an infective agent, e.g., tuberculosis). In particular, a composition of the disclosure can be used to treat (pre- or post-exposure) infection by bacteria, including Mycobacterium tuberculosis, Mycobacterium africanum, Mycobacterium avium, Mycobacterium bovis, Mycobacterium canetti, Mycobacterium chelonae, Mycobacterium fortuitum, Mycobacterium gordonae, Mycobacterium hiberniae, Mycobacterium intracellulare, Mycobacterium leprae, Mycobacterium kansasii, Mycobacterium marinum, Mycobacterium microti, Mycobacterium paratuberculosis, M. phlei, Mycobacterium pinnipedii, Mycobacterium scrofulaceum, Mycobacterium simiae, Mycobacterium smegmatis, Mycobacterium szulgai, Mycobacterium ulcerans, Mycobacterium vacca, Mycobacterium xenopi, Salmonella typhimurium, Escherichia coli, Klebsiella pneumoniae, Streptococcus pneumoniae, Staphylococcus aureus, Francisella tularensis, Brucella, Burkholderia mallei, Yersinia pestis, Corynebacterium diphtheria, Neisseria meningitidis, Bordetella pertussis, Clostridium tetani, or Bacillus anthracis; viruses of a viral family selected from the group containing Retroviridae, Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillomaviridae, Parvoviridae, Astroviridae, Polyomaviridae, Calciviridae, and Reoviridae; parasites, including Toxoplasma gondii, Plasmodium falciparum, Plasmodium vivax, Plasmodium ovate, Plasmodium malariae, Trypanosoma spp., or Legionella spp.; and fungi, including Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

Accordingly, in other non-limiting embodiments, a pharmaceutical composition of the disclosure can be used for treatment and/or prophylaxis of a subject (e.g., a human) with tuberculosis, leprosy, acquired immune deficiency syndrome (AIDS), cancer, typhoid fever, pneumonia, meningitis, staphylococcal scalded skin syndrome (SSSS), Ritter's disease, tularemia (rabbit fever), brucellosis, Glanders disease, bubonic plague, septicemic plague, pneumonic plague, diphtheria, pertussis (whooping cough), tetanus, anthrax, hepatitis, smallpox, monkeypox, measles, mumps, rubella, chicken pox, polio, rabies, Japanese encephalitis, herpes, mononucleosis, influenza, Ebola virus disease, hemorrhagic fever, yellow fever, Marburg virus disease, toxoplasmosis, malaria, trypanosomiasis, legionellosis, aspergillosis, blastomycosis, candidiasis (thrush), coccidioidomycosis, cryptococcosis, histoplasmosis, paracoccidioidomycosis, sporotrichosis, or sinus-orbital zygomycosis.

### Methods of Monitoring Treatment Responsiveness

Treatment responsiveness to an immunogenic composition of ΔLprG Mtb (e.g., a vaccine), other vaccines (e.g., BCG, MTBVAC, VPM1002, DAR-901, MVA85A, ChAdOx1.PPE15, TB/FLU-04L, Ad5Ag85A, AERAS-402, M72 (e.g., M72/AS01_{E}), RUTI, H107, or CysVac2/Advax), or other immunogenic compositions can be monitored by measuring one or more biomarkers (e.g., IL-17A, IL-6, IP-10, MIP-2, MIP-1α, MCP-1, IL-2, IL-10, IFNγ, TNFα, TNF-α-secreting CD4⁺ T cells, IFN-γ/TNF-α-secreting CD4⁺ T cells, PD-1-negative Ag-specific CD4⁺ T cells, and/or PD-1-positive Ag-specific CD4⁺ T cells) in a sample (e.g., blood (e.g., whole blood, serum, or plasma), bronchoalveolar lavage, or a lung biopsy) from a subject after administration of the immunogenic composition or vaccine. In some embodiments, the one or more biomarkers measured is a protein (e.g., a cytokine), nucleic acid (e.g., an RNA encoding a cytokine), or a cell (e.g., a T cell). In some embodiments, the one or more biomarkers measured is IL-17A, IL-6, IP-10, MIP-2, MIP-1α, MCP-1, IL-2, IL-10, IFNγ, TNFα, TNF-α-secreting CD4⁺ T cells, IFN-γ/TNF-α-secreting CD4⁺ T cells, PD-1-negative Ag-specific CD4⁺ T cells, and/or PD-1-positive Ag-specific CD4⁺ T cells (including combinations of two, three, four, or more of these biomarkers (e.g., two or more of IL-17A, IL-6, IP-10, and MIP-1α or two or more of IL-17A, IL-2, IL-10, IFNγ, and TNFα)). In some embodiments, the sample is a blood sample (e.g., a whole blood sample, a serum sample, or a plasma sample), a bronchoalveolar lavage sample, or a lung biopsy sample.

Biomarker levels in post-administration samples collected from a subject may be compared to biomarker levels in pre-administration samples collected from the subject or to a reference level of biomarker. The reference level of biomarker may be determined by identifying the biomarker levels present in samples from a reference population. The reference population may share one or more characteristics (e.g., health status, medical history, or other demographic data) with the subject or may be a historical control level.

A biomarker level in a post-administration sample collected from a subject that is greater than a biomarker level in a pre-administration sample or a reference biomarker level can be indicative of treatment responsiveness. An increase between at least about 1.01- and about 100-fold (e.g., 1.01-, 1.02-, 1.05-, 1.1-, 1.15-, 1.2-, 1.3-, 1.4-, 1.5-, 1.75-, 2-, 3-, 5-, 10-, 20-, 50-, or 100-fold), such as about 1.1-fold, in biomarker levels in post-administration samples compared to pre-administration samples can be indicative of treatment responsiveness. A biomarker level in a post-administration sample collected from a subject that is greater than the level present in between the about two hundredths percentile and about 100^{th} percentile (e.g., two hundredths, five hundredths, fifteen hundredths, two tenths, three tenths, four tenths, five tenths, 1^{st}, one and five tenths, 2^{nd}, 3^{rd}, 4^{th}, 5^{th}, 6^{th}, 7^{th}, 10^{th}, 15^{th}, 20^{th}, 25^{th}, 30^{th}, 40^{th}, 50^{th}, 60^{th}, 68^{th}, 70^{th}, 75^{th}, 80^{th}, 82 and five tenths, 90^{th}, 95^{th}, 98^{th}, 99^{th}, 99 and five tenths, 99 and seven tenths, 99 and eight tenths, 99 and 85 hundredths, 99 and nine tenths, 99 and 95 hundredths, 99 and 98 hundredths, or 100^{th} percentile), such as the about 50^{th} percentile, of a reference population can be indicative of treatment responsiveness. Preferably, a biomarker level in a post-administration sample collected from the subject that is greater than the level present in the 75^{th} percentile of a naïve reference population or greater than the level present in the three tenths percentile of a post-vaccination or a ΔLprG Mtb post-administration reference population can be indicative of treatment responsiveness. In some embodiments the reference biomarker level is a protein level (e.g., an IL-17A, IL-6, IP-10, MIP-2, MIP-1α, MCP-1, IL-2, IL-10, IFNγ, or TNFα protein level). The reference biomarker level for IL-17A may be about 5 pg/mL (e.g., about 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, or more). The reference biomarker level for IL-6 may be about 10 pg/mL (e.g., about 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 25 pg/mL, 30 pg/mL, or more). The reference biomarker level for IFNγ may be about 10 pg/mL (e.g., about 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 25 pg/mL, 30 pg/mL, or more). The reference biomarker level for MIP-2 may be about 150 pg/mL (e.g., about 150 pg/mL, 160 pg/mL, 170 pg/mL, 180 pg/mL, 190 pg/mL, 200 pg/mL, 225 pg/mL, 250 pg/mL, 275 pg/mL, or more). The reference biomarker level for IP-10 may be about 200 pg/mL (e.g., about 200 pg/mL, 225 pg/mL, 250 pg/mL, 275 pg/mL, 300 pg/mL, 325 pg/mL, 350 pg/mL, 375 pg/mL, 400 pg/mL, 410 pg/mL, 420 pg/mL, 430 pg/mL, 440 pg/mL, 450 pg/mL, 480 pg/mL, 500 pg/mL, 550 pg/mL, 600 pg/mL, 700 pg/mL, 800 pg/mL, 900 pg/mL, 1000 pg/mL, or more). The reference biomarker level for G-CSF may be about 800 pg/mL (e.g., about 800 pg/mL, 850 pg/mL, 900 pg/mL, 950 pg/mL, 1000 pg/mL, 1200 pg/mL, 1500 pg/mL, 1750 pg/mL, 2000 pg/mL or more). The reference biomarker level for MCP-1 may be about 30 pg/mL (e.g., about 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 45 pg/mL, 50 pg/mL, or more). The reference biomarker level for MIP-1α may be about 80 pg/mL (e.g., about 80 pg/mL, 81 pg/mL, 82 pg/mL, 83 pg/mL, 84 pg/mL, 85 pg/mL, 86 pg/mL, 87 pg/mL, 88 pg/mL, 89 pg/mL, 90 pg/mL, 95 pg/mL, 100 pg/mL, 110 pg/mL, 120 pg/mL, or more).

In some embodiments, multiple biomarker levels from multiple samples may be combined or compared. Multiple pre- or post-administration samples may be collected to monitor biomarker levels over time, such as between about 1 minute to about 12 weeks (e.g., e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, or 12 weeks). For example, peak post-administration biomarker levels, average biomarker levels, or the combined biomarker levels of multiple samples may be compared to one or more pre-administration biomarker levels or reference biomarker levels. Multiple samples may also be collected to account for normal variation in biomarker levels.

A biomarker level in a post-administration sample that is not increased relative to a pre-administration sample or is not greater than a reference biomarker level can indicate that the subject did not respond to treatment and is in need of re-administration of the immunogenic composition of ΔLprG Mtb or other vaccine at the same or a different dose by the same or a different route of administration. Thus, the methods of treatment or prophylaxis described above may be repeated in a subject when the subject's biomarker levels indicate an insufficient or incomplete immune response against the ΔLprG Mtb composition or other vaccine composition.

In some embodiments, treatment responsiveness to an immunogenic composition of ΔLprG Mtb (e.g., a vaccine), other vaccines (e.g., BCG, MTBVAC, VPM1002, DAR-901, MVA85A, ChAdOx1.PPE15, TB/FLU-04L, Ad5Ag85A, AERAS-402, M72 (e.g., M72/AS01_{E}), RUTI, H107, or CysVac2/Advax), or other immunogenic compositions can be monitored by measuring the presence of IL-17A (e.g., IL-17A protein) in a sample (e.g., blood, such as serum or plasma) from a subject after administration of the immunogenic composition or vaccine. IL-17A levels may also be measured by measuring IL-17A⁺ cells (e.g., IL-17A⁺ helper T cells) in a sample (e.g., bronchoalveolar lavage, a lung biopsy, or blood) from the subject.

Samples may be taken from a subject prior to administration (henceforth referred to as “pre-administration”) of an immunogenic composition or vaccine, such as, between about 1 minute to about 12 weeks (e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, or 12 weeks), such as about 5 minutes, prior to administration, to establish a baseline level of one or more of the biomarkers noted above (e.g., IL-17A) to which later samples may be compared. Samples may be taken from a subject after administration of an immunogenic composition or vaccine, such as, between about 1 minute to about 12 weeks (e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, or 12 weeks), such as about 2 weeks, after administration (henceforth referred to as “post-administration”).

IL-17A levels in post-administration samples collected from a subject may be compared to IL-17A levels in pre-administration samples collected from the subject or to a reference level of IL-17A. The reference level of IL-17A may be determined by identifying the IL-17A levels present in samples from a reference population. Preferably, the reference population will share one or more characteristics (e.g., health status, medical history, or other demographic data) with the subject or may be a historical control level.

An IL-17A level in a post-administration sample collected from a subject that is greater than an IL-17A level in a pre-administration sample or a reference IL-17A level can be indicative of treatment responsiveness. An increase between at least about 1.01- and about 100-fold (e.g., 1.01-, 1.02-, 1.05-, 1.1-, 1.15-, 1.2-, 1.3-, 1.4-, 1.5-, 1.75-, 2-, 3-, 5-, 10-, 20-, 50-, or 100-fold), such as about 1.1-fold, in IL-17A levels in post-administration samples compared to pre-administration samples can be indicative of treatment responsiveness. An IL-17A level in a post-administration sample collected from a subject that is greater than the level present in between the about two hundredths percentile and about 100^{th} percentile (e.g., two hundredths, five hundredths, fifteen hundredths, two tenths, three tenths, four tenths, five tenths, 1^{st}, one and five tenths, 2^{nd}, 3^{rd}, 4^{th}, 5^{th}, 6^{th}, 7^{th}, 10^{th}, 15^{th}, 20^{th}, 25^{th}, 30^{th}, 40^{th}, 50^{th}, 60^{th}, 68^{th}, 70^{th} 75^{th} 80^{th}, 82 and five tenths, 90^{th}, 95^{th}, 98^{th}, 99^{th}, 99 and five tenths, 99 and seven tenths, 99 and eight tenths, 99 and 85 hundredths, 99 and nine tenths, 99 and 95 hundredths, 99 and 98 hundredths, or 100^{th} percentile), such as the about 50^{th} percentile, of a reference population can be indicative of treatment responsiveness. Preferably, an IL-17A level in a post-administration sample collected from the subject that is greater than the level present in the 75^{th} percentile of a naïve reference population or greater than the level present in the three tenths percentile of a post-vaccination or a ΔLprG Mtb post-administration reference population can be indicative of treatment responsiveness. The reference biomarker level for IL-17A may be about 5 pg/mL (e.g., about 5 pg/mL, 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, or more).

In some embodiments, multiple IL-17A levels from multiple samples may be combined or compared. Multiple pre- or post-administration samples may be collected to monitor IL-17A levels over time, such as between about 1 minute to about 12 weeks (e.g., e.g., 1 minute, 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 20 hours, 1 day, 2 days, 3 days, 4 days, 5 days, 6 days, 1 week, 2 weeks, 4 weeks, 8 weeks, or 12 weeks). For example, peak post-administration IL-17A levels, average IL-17A levels, or the combined IL-17A levels of multiple samples may be compared to one or more pre-administration IL-17A levels or reference IL-17A levels. Multiple samples may also be collected to account for normal variation in IL-17A levels.

An IL-17A level in a post-administration sample that is not increased relative to a pre-administration sample or is not greater than a reference IL-17A level can indicate that the subject did not respond to treatment and is in need of re-administration of the immunogenic composition of ΔLprG Mtb or other vaccine at the same or a different dose by the same or a different route of administration. Thus, the methods of treatment or prophylaxis described above may be repeated in a subject when the subject's IL-17A levels indicate an insufficient or incomplete immune response against the ΔLprG Mtb composition or other vaccine composition.

### Kits Containing ΔLprG Mtb

Described herein are immunogenic compositions of ΔLprG Mtb, methods of inducing an immune response by administering immunogenic compositions of ΔLprG Mtb, and monitoring responsiveness to immunogenic compositions. Although each aspect may be practiced separately, reagents for practicing one or more of these methods may also be incorporated into a kit. For example, a kit may be sold with one or more immunogenic compositions of ΔLprG Mtb and one or more reagents (e.g., an antibody, such as an anti-IL-17A antibody) for detection of one or more of the biomarkers described herein (e.g., IL-17A). In some embodiments, the kit may also include instructions for use and/or one or more other immunogenic compositions (e.g., BCG, MTBVAC, VPM1002, DAR-901, MVA85A, ChAdOx1.PPE15, TB/FLU-04L, Ad5Ag85A, AERAS-402, M72 (e.g., M72/AS01_{E}), RUTI, H107, or CysVac2/Advax). Based on the disclosure provided herein, one of ordinary skill in the art will recognize a multitude of possible kit combinations.

The following are examples of the methods of the disclosure. It is understood that various other aspects may be practiced, given the general descriptions provided above.

### IV. Examples

### Example 1. Use of Attenuated Mtb ΔLprG Vaccine to Induce an Immune Response in a Mammal

The ΔLprG vaccine can be formulated as a whole cell vaccine based on an Mtb strain, which has been genetically engineered to delete key virulence factors and potential immune evasion genes. The ΔLprG vaccine induced higher antigen-specific cytokine-secreting CD4⁺ T lymphocytes in peripheral blood and lung than BCG and markedly improved protective efficacy against both homologous and heterologous aerosolized Mtb challenges in C3HeB/FeJ mice.

Mice, Immunizations, and Mycobacterium tuberculosis Infections

Female 6-10-wk-old C57BL/6J, Balb/cJ, SCID, and C3HeB/FeJ mice (The Jackson Laboratory, Bar Harbor, Me.) were housed under sterile conditions in an ABSL3 facility, and all animal experiments were performed under an animal protocol approved by Harvard University. Mycobacterial strains were grown in 7H9 with 10% (vol/vol) OADC (Middlebrook), 0.2% glycerol and Tween 80 and maintained at 37° C. with shaking at 100 rpm unless otherwise indicated. Mycobacterial strains for vaccines lots were prepared as previously described with minor modifications (Hart et al. 22, 726-741 (2015)). Cultures of Bacillus Calmette-Guérin (BCG)-Danish (BCG SSI) originally obtained from Statens Serum Institute (Copenhagen, Denmark) and Mtb H37Rv A/prG-rv1410c (ΔLprG; Martinot et. al. 2016) vaccines were expanded in 7H9 with 10% OADC, 0.5% glycerol, and 0.05% Tyloxapol to a 100 mL volume in roller bottles. Log phase cultures were pelleted by centrifugation at 2000 rcf for 15 min and washed with equal volume (1:1) of PBS with 0.05% Tyloxapol, re-centrifuged followed by wash with 50% volume (1:2) of PBS-0.05% Tyloxapol, then re-centrifuged and resuspended in 25% volume (1:4) of PBS-0.05% Tyloxapol with 15% glycerol. To remove clumps, 10 mL volumes of bacterial suspension were filtered twice, first through 40 μm then 20 μm vacuum filter units (Millipore). The optical density 600 (OD600) of a 1:10 dilution of the resultant suspension was measured and then the filtrate was back diluted to OD 1.0 or OD 5.0 with additional of PBS-0.05% Tyloxapol with 15% glycerol. Mice received immunizations of 100 uL of OD 1.0 bacterial culture (2⁺/−1×10⁷ CFU/mL) in PBS-0.05% Tyloxapol with 15% glycerol subcutaneously in the left flank of either BCG SSI or ΔLprG. Some mice received H37Rv prepared in a similar fashion as a control for immunogenicity studies. For dose finding experiments, C57BL/6J mice were vaccinated with BCG Pasteur or ΔLprG from freshly propagated vaccine cultures. Mice were rested a minimum of 8 weeks post-vaccination prior to aerosol challenge with 75⁺/−25 CFU of either Mycobacterium tuberculosis H37Rv or Erdman.

Tissue Processing and CFU Enumeration

In all studies, lungs from Mtb challenged mice were aseptically collected 4 weeks following Mtb challenge and perfused with PBS prior to tissue harvest to remove red blood cells by transection of the abdominal aorta followed by injection of the right ventricle with 10 mL cold sterile phosphate buffered saline. The three right lung lobes were used to enumerate CFU and were homogenized in sterile 1×PBS, followed by serial dilutions onto 7H10 plates and incubated for 3 weeks at 37° C. The left lung lobe was collected into RPMI with 10% fetal bovine serum (FBS) and homogenized using scissors for lung leukocyte isolation. Lung homogenate was incubated for 30 min in digestion buffer containing RPMI, FBS, Type IV collagenase (Sigma C5138) and DNase at 37° C. with gentle rocking. Lung digests were filtered through 30 μm MACS SmartStrainers (Miltenyi Biotec) and washed with fresh media and resuspended in a standardized volume of 6 mL media prior to plating in 96 well U-bottom plates at an approximate density of 2×10⁶ cells per well. In some cases, the accessory lobe was inflated with 100 uL of 10% neutral buffered formalin for histopathology.

Luminex Assays for Cytokine Secretion

Serum samples were collected at days 1, 8 (+/−1 day), and 14 days (+/−1 day) post vaccination and compared to non-vaccinated Naïve mice. Samples were filtered twice through 0.2 micron 96 well filtration plates by centrifugation (Millipore), treated with 0.05% Tween-20, and assayed using a Luminex bead-based multiplex ELISA (MILLIPLEX MAP Mouse Cytokine/Chemokine Magnetic Bead Panel (Millipore; MCYTMAG-70K-PX32) according to the manufacturer's instructions. Samples were fixed with 4% formaldehyde and subsequently washed prior to acquisition. Sample data were acquired on a MAGPIX instrument running xPONENT 4.2 software (Luminex Corp.) and analyzed using a five-parameter logistic model with an 80-120% standard acceptance range. The MILLIPLEX MAP Mouse High Sensitivity T cell panel (Millipore; MHSTCMAG-70K) was used to evaluate low levels of IL-17A. <LLOQ indicates lower limit of quantification for assay; extrapolated values below the LLOQ were evaluated at the LLOQ. Multiple regression analyses of cytokines serum levels with CFU in lung and spleen were performed using the R mixOmics package (Rohart et al. PLoS Comput Biol. 13, e1005752 (2017)). Heatmaps of serum cytokine and chemokine levels in lung and spleen were generated using R heatmap package.

Flow Cytometry and Intracellular Cytokine Staining (ICS)

Lymphocytes were isolated from either blood, spleen, or lung, stained, and analyzed by flow cytometry as previously described (Provine et al., The Journal of Immunology. 192, 5214-5225 (2014)). Antibodies (Ab) to CD8a (53-6.7), CD4 (RM4-5), CD44 (IM7), PD-1 (RMPI-30), IFN-γ (XMG1.2), TNF-α (MP6-XT22), IL-2 (JES6-5H4), IL-17 (TC11-18H10), and IL-10 (JES5-16E3) were purchased from BD Biosciences (Myrtle, U.K.), eBioscience, or BioLegend (San Diego, Calif.). Cell viability was assessed by LIVE/DEAD Fixable Aqua (Life Technologies). Ag-specific cells were estimated by intracellular cytokine staining (ICS). Peripheral blood mononuclear cells (PBMC), splenocytes, or lung leukocytes were stimulated with a 1:200 dilution of purified protein derivative (PPD, Tuberculin OT, Synbiotics, Corp, San Diego) and incubated for 40 min at 37° C. After this incubation, Golgi-Plug and Golgi-Stop (BD Biosciences) were added and samples incubated for an additional 6.5 hours at 37° C. Cells were subsequently washed and stained for surface antibody markers, then permeabilized with Cytofix/Cytoperm (BD Biosciences) and stained for intracellular cytokines. Cells were acquired on an LSR II flow cytometer (BD Biosciences). Data were analyzed using FlowJo v10.

Histopathology and Image Analysis

Lungs from infected mice were inflated with 10% neutral buffered formalin, processed, embedded in paraffin, and sectioned for staining. Formalin-fixed paraffin embedded (FFPE) serial tissue sections were stained with hematoxylin and eosin (H&E) and Ziehl-Neelsen acid-fast stains. Scoring for percent lung affected and acid-fast staining per granuloma was performed by two independent veterinary pathologists. Slides were digitized and lung granuloma area (mm²) quantified using Aperio Imagescope (Leica Biosystems).

Statistical Analysis

Statistical analyses were performed using Prism 8.0 (GraphPad Software). Data were analyzed by the Kruskal-Wallis test with Dunn multiple comparison post-test (more than two groups) or the two-tailed Mann-Whitney U test (for two groups). Longitudinally acquired Luminex data were analyzed using a two-tailed Mann-Whitney U test for each time point; not corrected for multiple comparisons across time points. Receiver operator curves (ROC) were generated using the ROC to baseline method with IL-17 values from the ultrasensitive Luminex assay (Yu et al. (Human Vaccines & Immunotherapeutics 14, 2692-2700, 2018)).

The ΔLprG Vaccine Protects Against Mtb Challenge in Mice and has a Comparable Attenuation to BCG

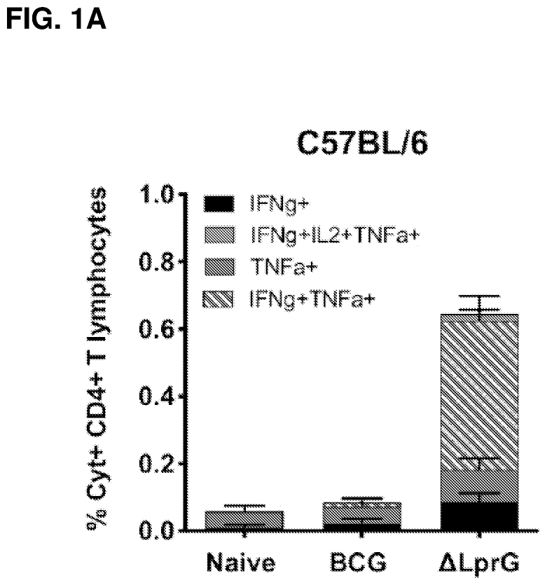

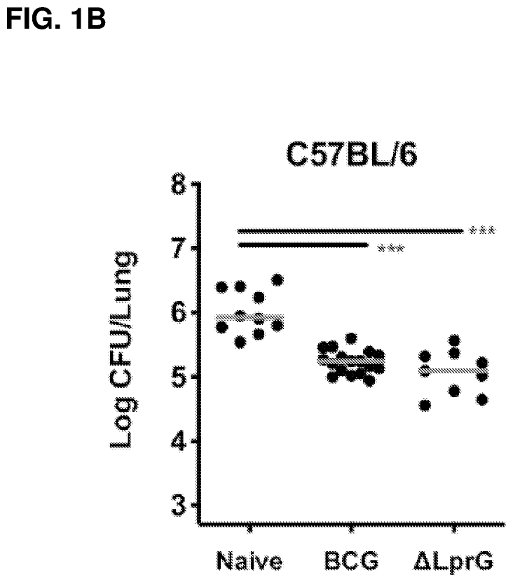

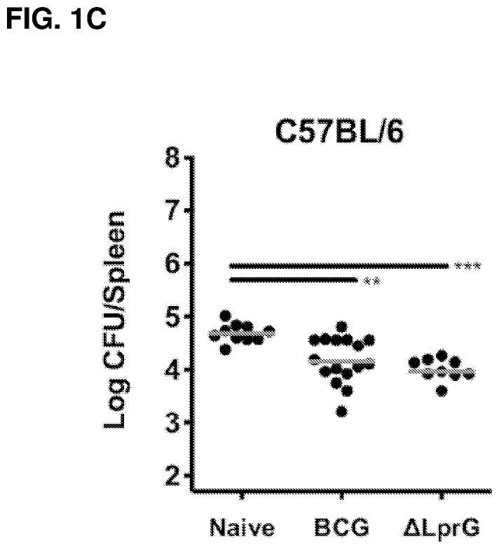

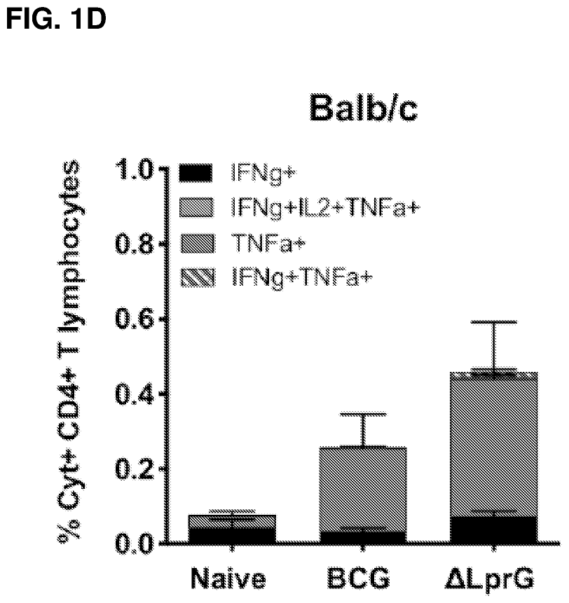

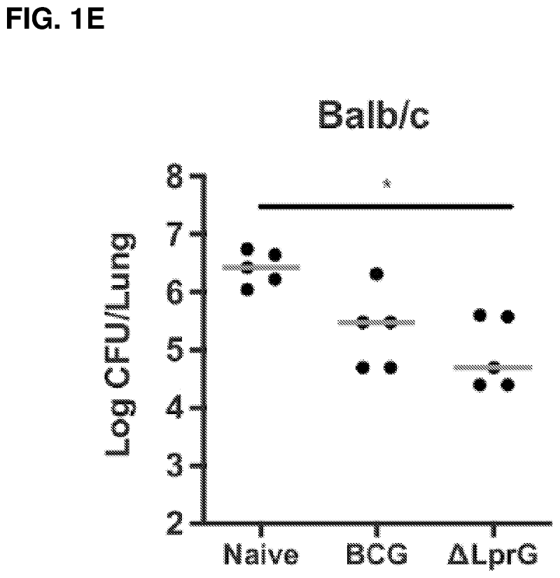

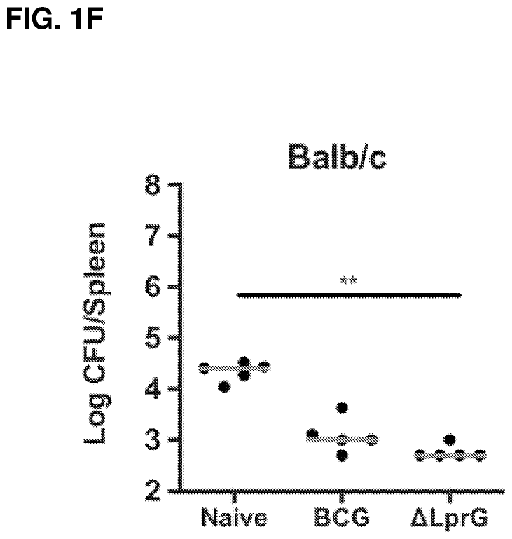

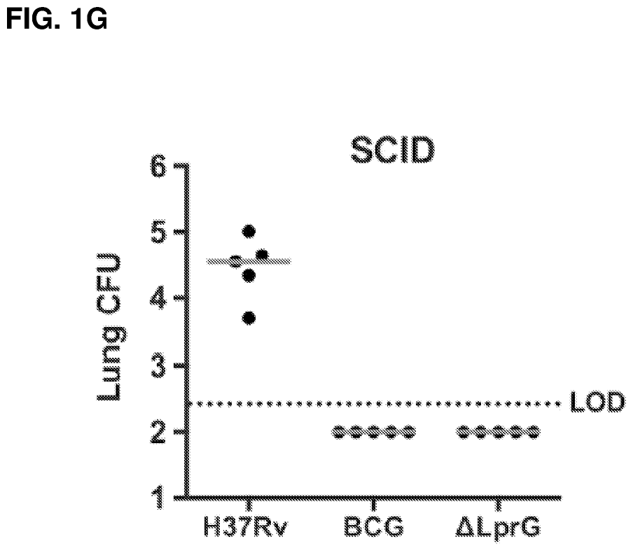

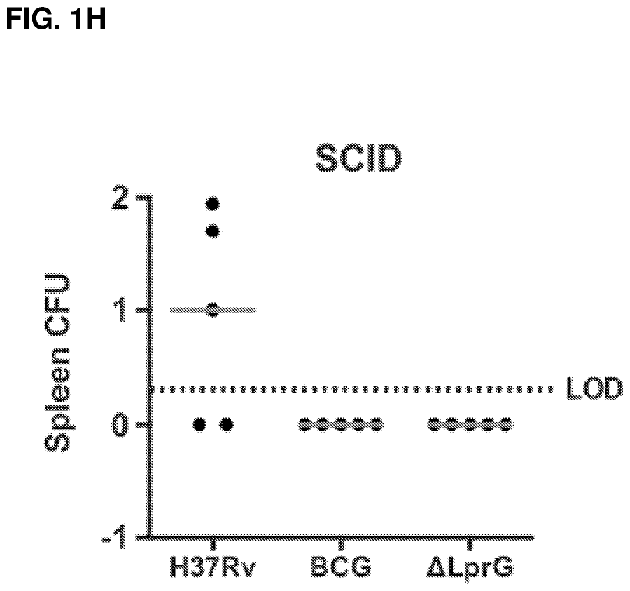

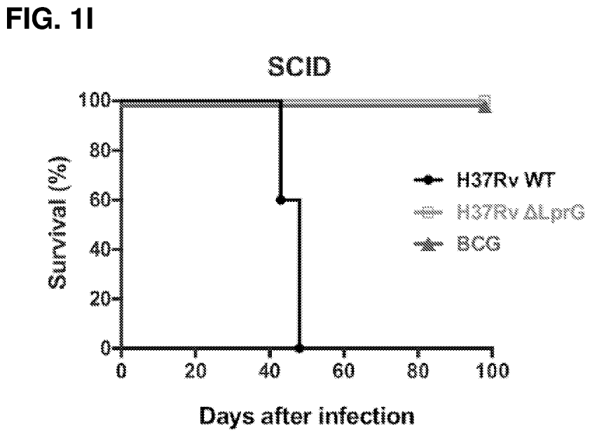

We evaluated the use of the previously characterized H37RvΔlprG-rv1410 (ΔLprG) deletion strain (Martinot et al. PLoS Pathog 12, e1005351 (2016)) as an alternative whole cell vaccine to BCG. We determined that ΔLprG induced Ag-specific T cells in peripheral blood of both C57BL/6J and Balb/cJ mice and tested the protective efficacy of the ΔLprG mutant as a vaccine strain in these backgrounds (FIGS. 1A-1F). Both ΔLprG and BCG vaccinated C57BL/6 (FIGS. 1A-1C) and Balb/cJ mice (FIGS. 1D-1F) demonstrated immunogenicity and reduced bacterial loads as compared to non-vaccinated mice (Naïve) after Mtb challenge. The ΔLprG deletion strain has been shown to be significantly attenuated in SCID mice as compared to WT and complemented mice after IV inoculation (Martinot et al. PLoS Pathog 12, e1005351 (2016). We further confirmed the attenuation phenotype with an aerosol administration of the ΔLprG vaccine in SCID mice, which showed a similar attenuation profile to BCG (FIGS. 1G, 1H). All BCG and ΔLprG vaccinated mice survived >100 days, whereas all Mtb infected mice died by day 50 (FIG. 1I).

ΔLprG Vaccination Leads to Enhanced Immunogenicity and Protection in Mice that Develop Necrotizing Granulomas

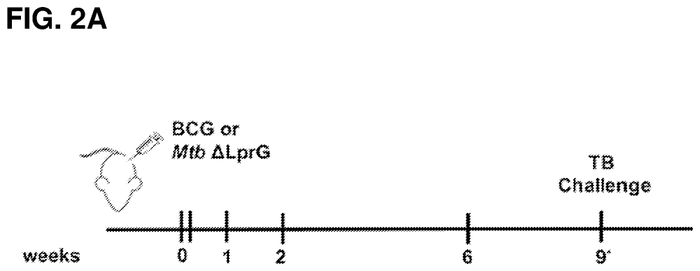

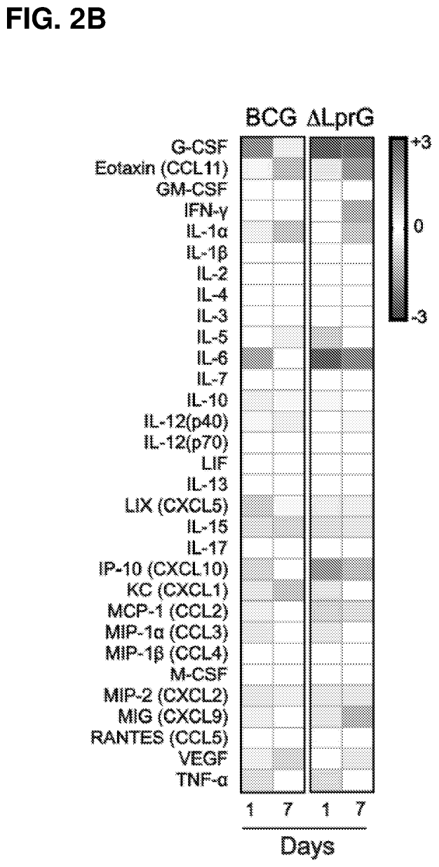

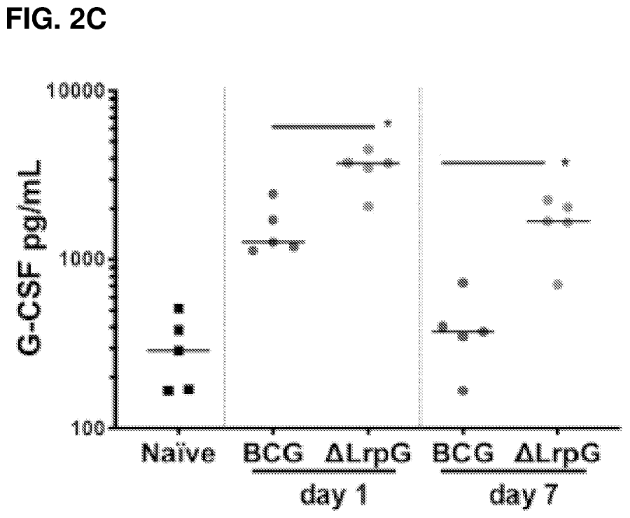

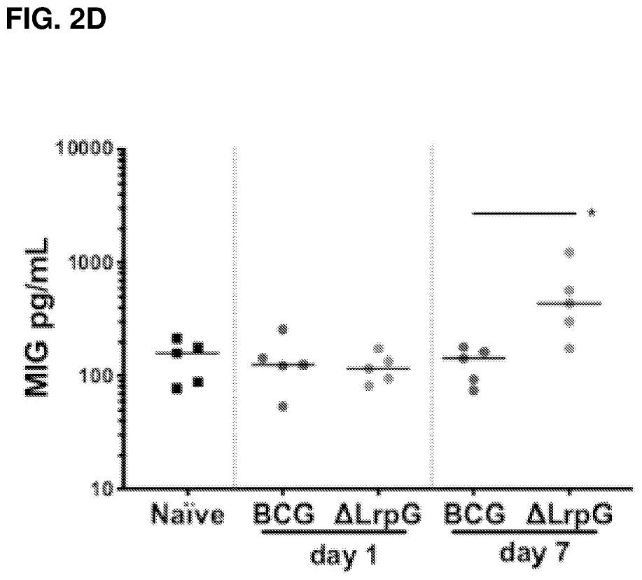

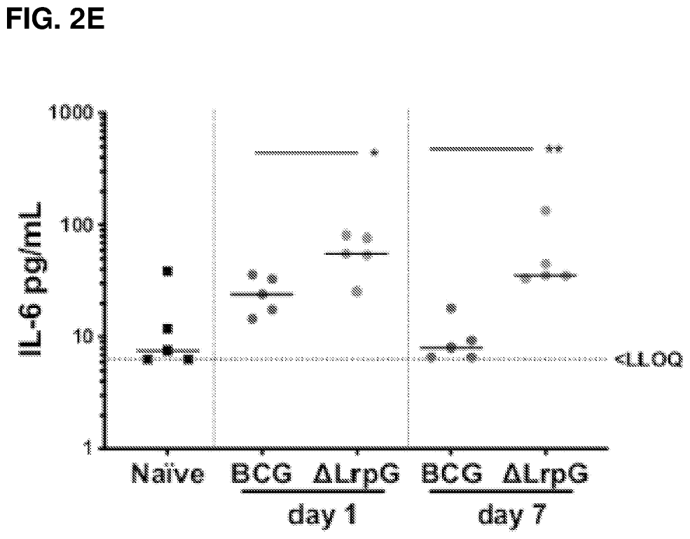

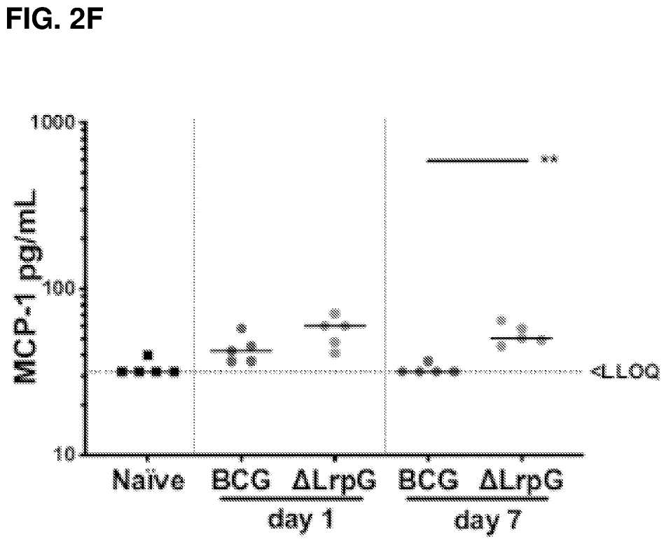

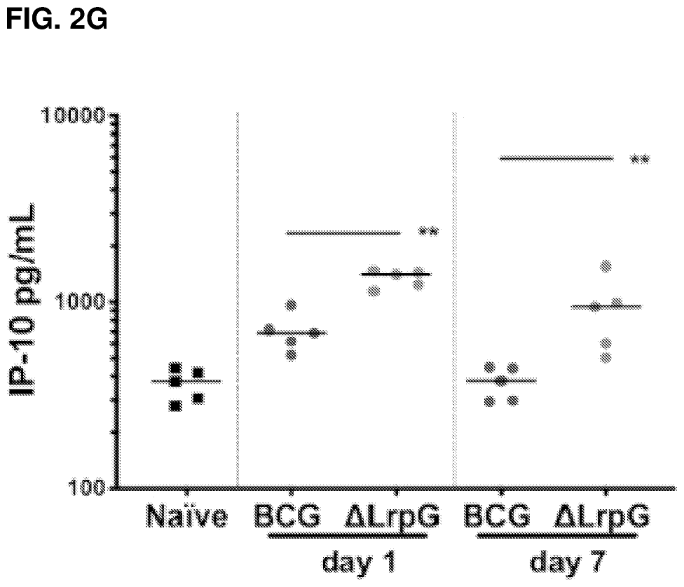

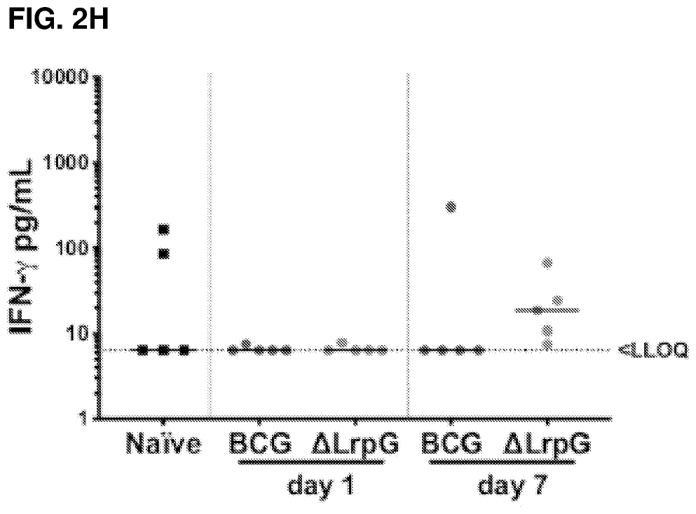

We tested the immunogenicity of BCG and ΔLprG vaccines in C3HeB/FeJ mice, which develop necrotizing granulomas that are similar to human TB granulomas. C3HeB/FeJ mice have a genetic susceptibility locus sst1 that renders them highly susceptible to tuberculosis disease (Pan et al. Nature. 434, 767-772 (2005)), and they develop lesions with central necrosis characterized by neutrophilic infiltrates that can develop into caseous and hypoxic lesions over time (Irwin et al. Dis Model Mech. 8, 591-602 (2015)) (Harper et al. J Infect Dis 205, 595-602 (2012)). Furthermore, we assessed vaccine immunogenicity longitudinally in peripheral blood of mice. C3HeB/FeJ mice were vaccinated by the subcutaneous route with 2×10⁷ colony-forming units (CFU) of either BCG or ΔLprG, formulated in phosphate-buffered saline with tyloxapol and glycerol (Hart et al. Clinical and Vaccine Immunology. 22, 726-741 (2015)). Serum was collected for Luminex analysis of cytokine and chemokine responses on days 0, 1, and 7 following vaccination, and PBMC were isolated at weeks 2, 6, and 9 (FIG. 2A). ΔLprG vaccination resulted in greater levels of serum inflammatory cytokines G-CSF (p=0.0159), IL-6 (p=0.0317), and IP-10 (p=0.0079) than BCG on day 1 and day 7 (p=0.0159, p=0.0079, p=0.0079, respectively) following vaccination (FIGS. 2B-2H). ΔLprG vaccination also led to greater serum MIG (p=0.0159) and MCP-1 (p=0.0079) than BCG on day 7 (FIGS. 2D, 2F).

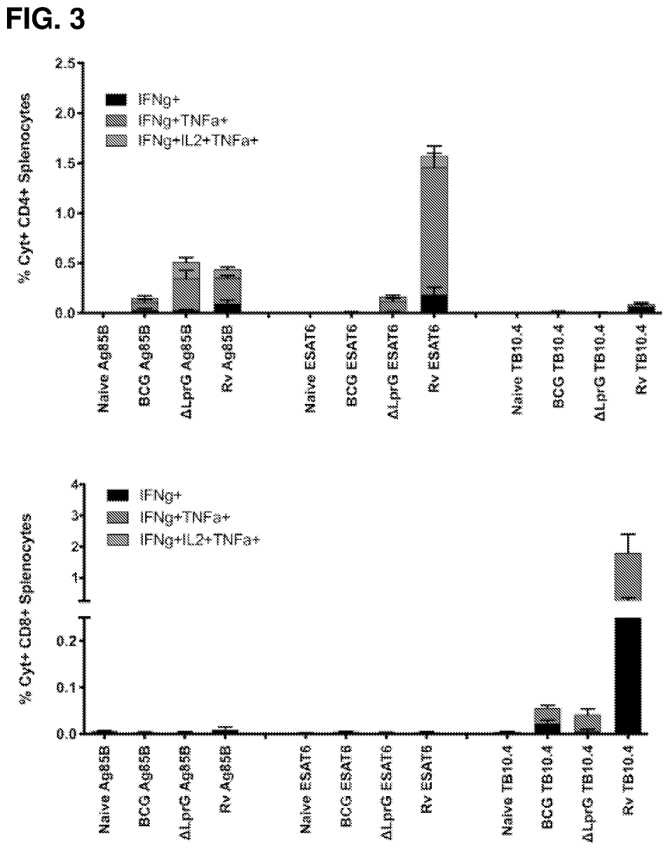

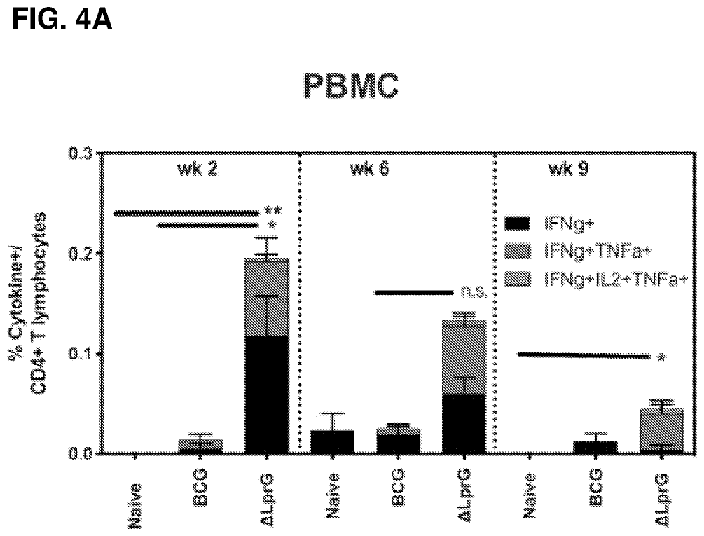

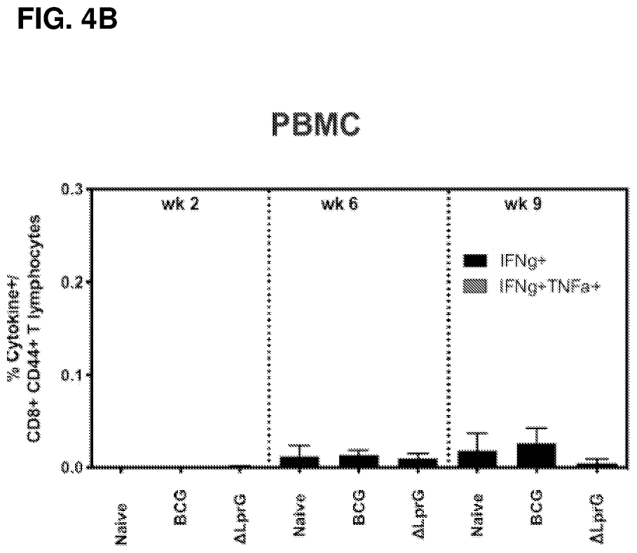

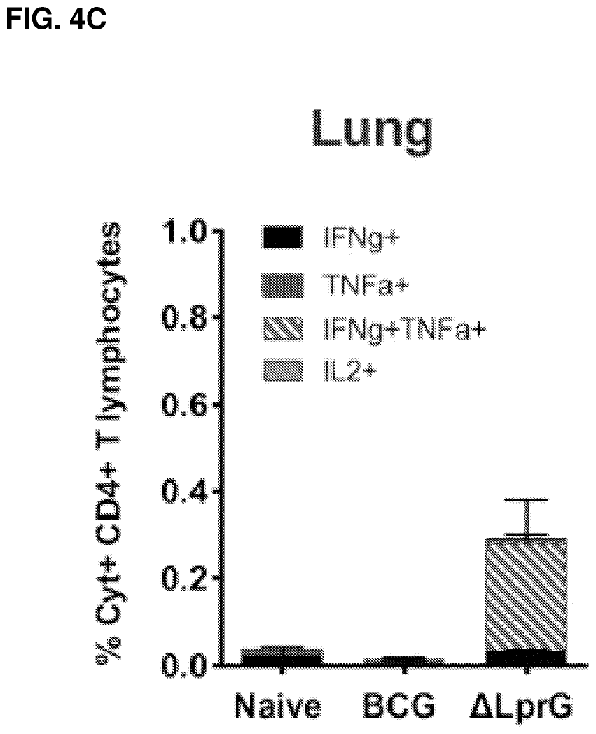

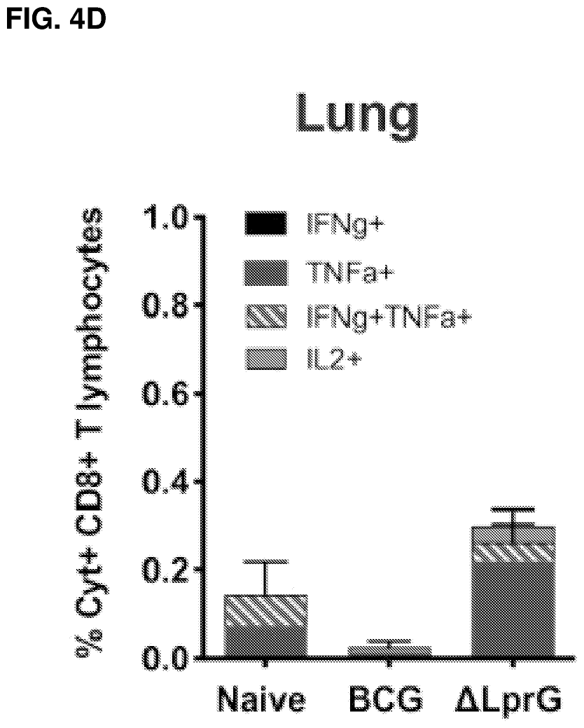

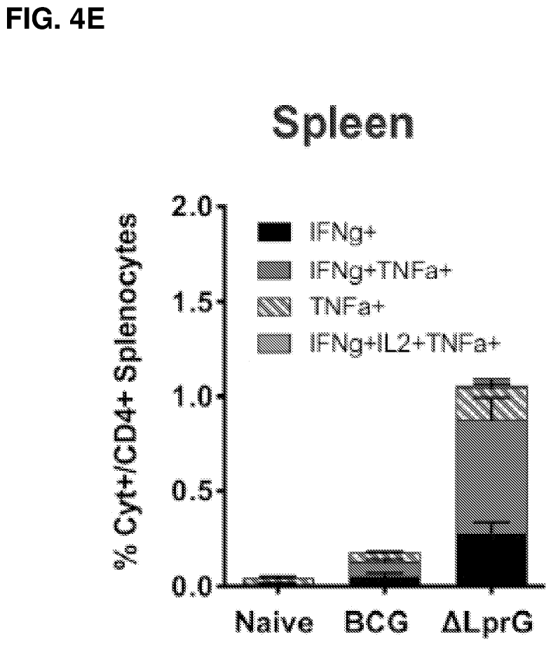

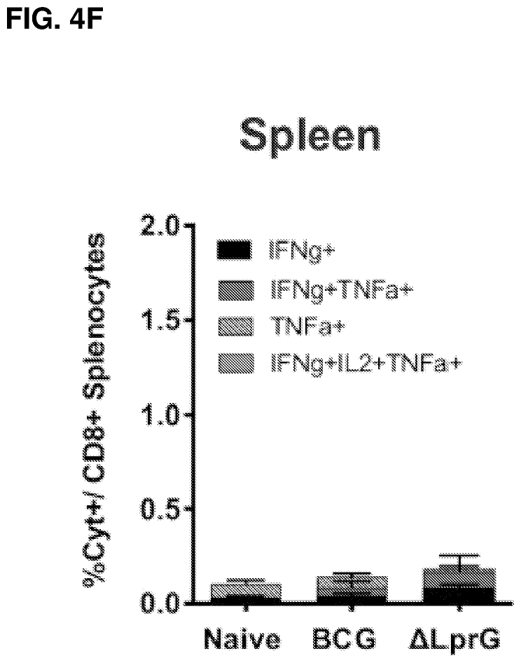

We next evaluated Ag-specific T lymphocyte responses in peripheral blood. ESAT-6, Ag85B, and TB10.4 are thought to potentially play a role in both innate immune signaling and protective immunity to TB (Hoang et al., PLoS ONE 8, e80579 (2013)) (Groschel et al. Cell Rep 18, 2752-2765 (2017)) (Aguilo et al. Nature Communications. 8, 16085 (2017)). Ag85B and ESAT-6 specific CD4+ T lymphocytes were detected in C57BL/6J mice exposed to Mtb lacking the LprG-Rv1410 operon, although at significantly lower levels than those induced by virulent Mtb, presumably due to the lack of replication of the mutant Mtb as compared to wild-type H37Rv (FIG. 3). On the contrary, C3HeB/FeJ mice are H2-k restricted, and Ag85B and ESAT-6 specific responses were below the limit of detection by intracellular cytokine staining. Therefore, PBMC were stimulated with purified protein derivative (PPD; Tuberculin OT, Synbiotic) and were assessed by ICS assays. Higher IFN-γ-, TNF-α-, and IL-2-secreting CD4⁺ T cell responses were observed in the blood of ΔLprG vaccinated mice as compared to BCG vaccinated mice at week 2 following vaccination (FIG. 4A; p=0.004), and these differences persisted at week 6 and week 9. In contrast, Ag-specific CD8⁺ T cell responses were comparable between ΔLprG and BCG vaccinated mice (FIG. 4B) in blood. ΔLprG mice also demonstrated increased numbers of Ag-specific CD4+ T lymphocytes in lung (FIG. 4C) and spleen (FIG. 4E) 2 weeks post-vaccination. Similar to blood, minimal differences were noted in lung and splenic CD8+T lymphocyte populations post-vaccination (FIGS. 4D, 4F).

At week 9, mice were challenged by the aerosolized route with 75 colony-forming units (CFU) of Mtb H37Rv and were sacrificed at week 4 following challenge to assess protective efficacy and immune correlates of protection. Prior to tissue harvest, lungs were perfused with phosphate-buffered saline (PBS) to clear peripheral erythrocytes and leukocytes from the pulmonary vasculature. Lungs were collected and homogenized, and pulmonary T cells were purified and stimulated with PPD and analyzed by ICS assays for IFN-γ, TNF-α, IL-2, IL-17, and IL-10.

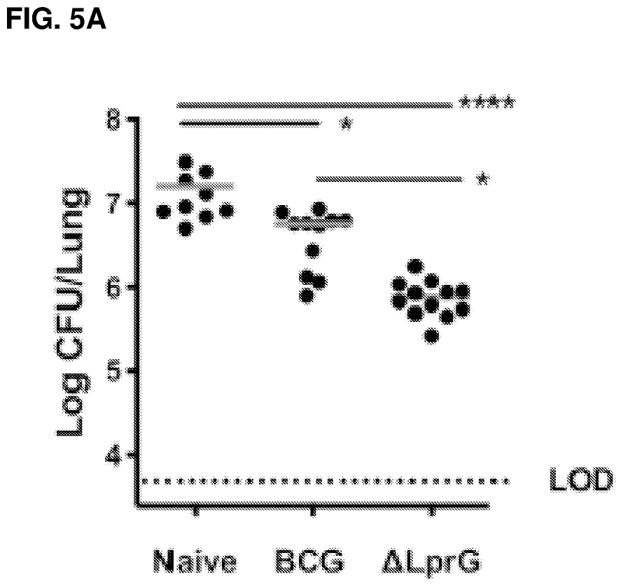

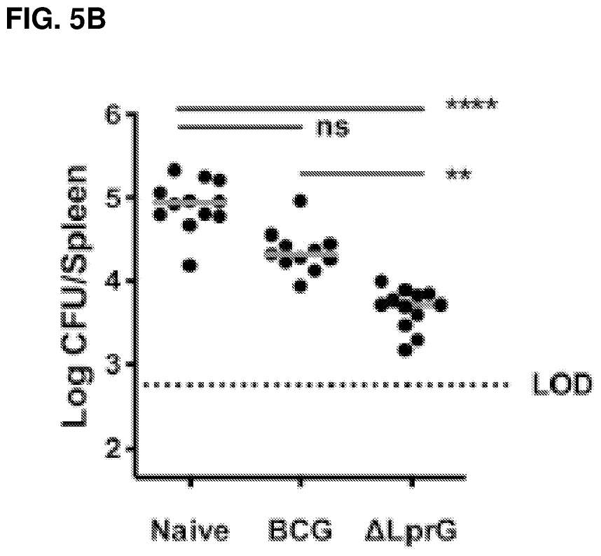

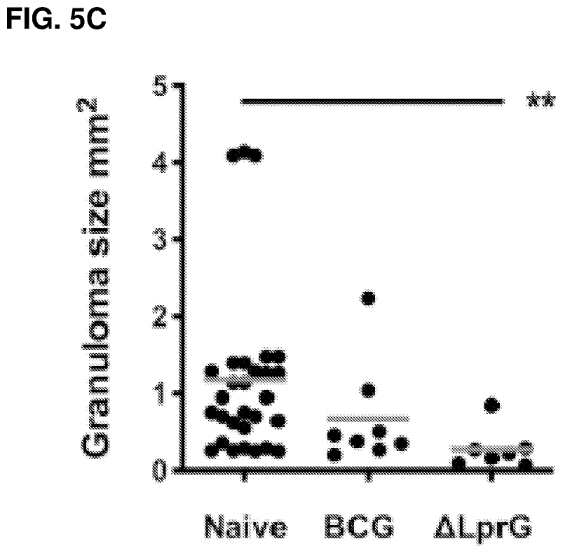

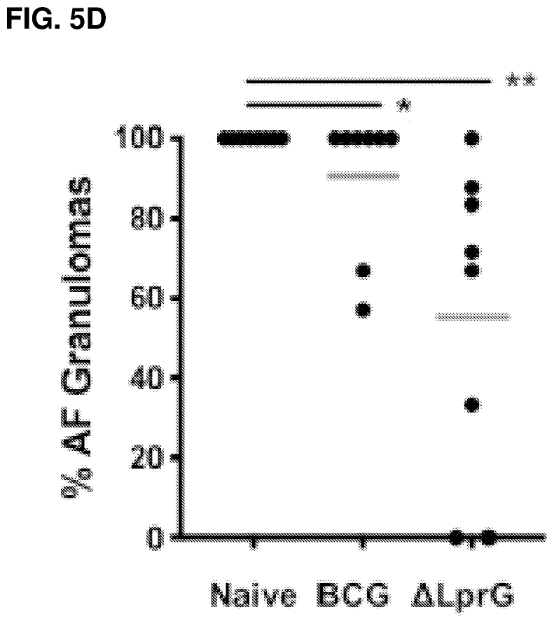

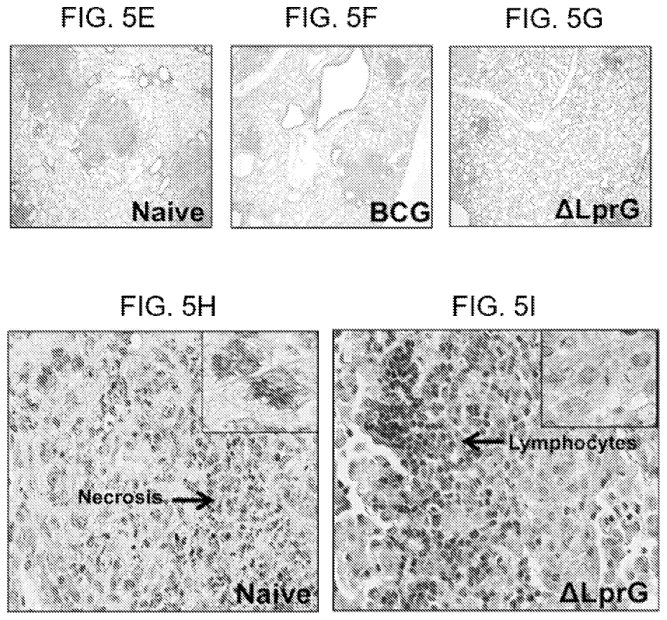

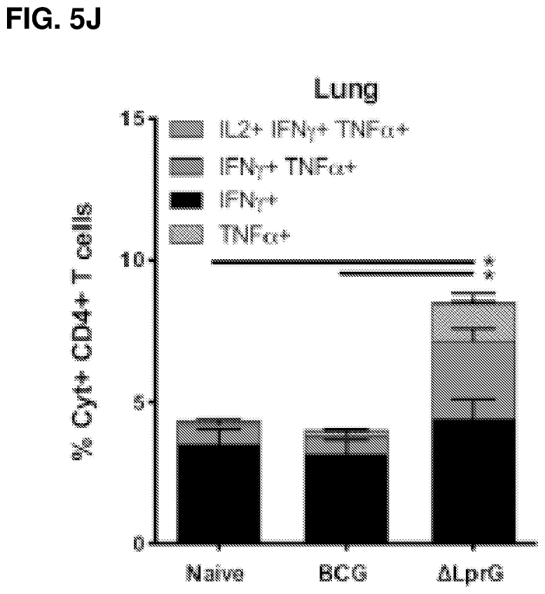

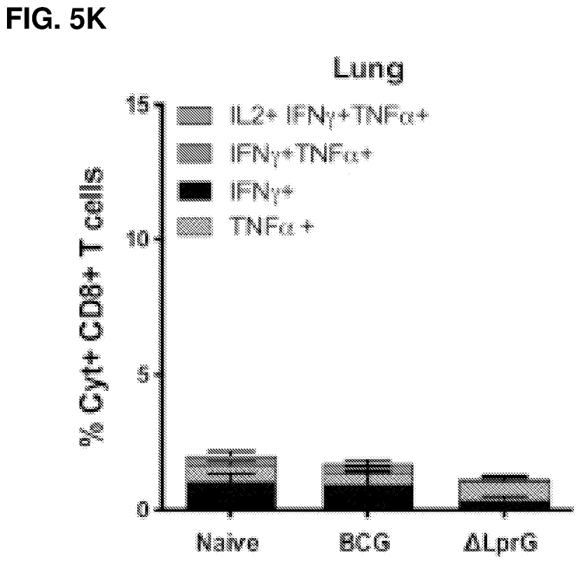

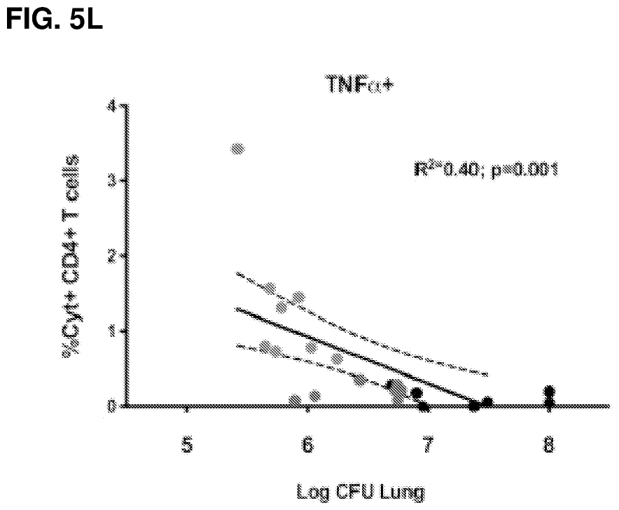

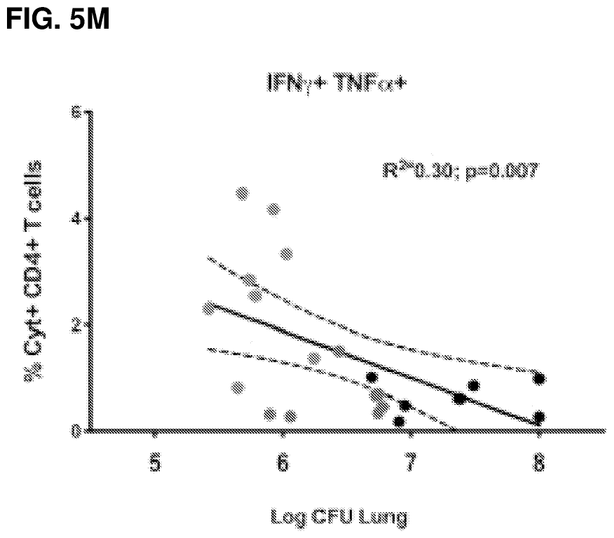

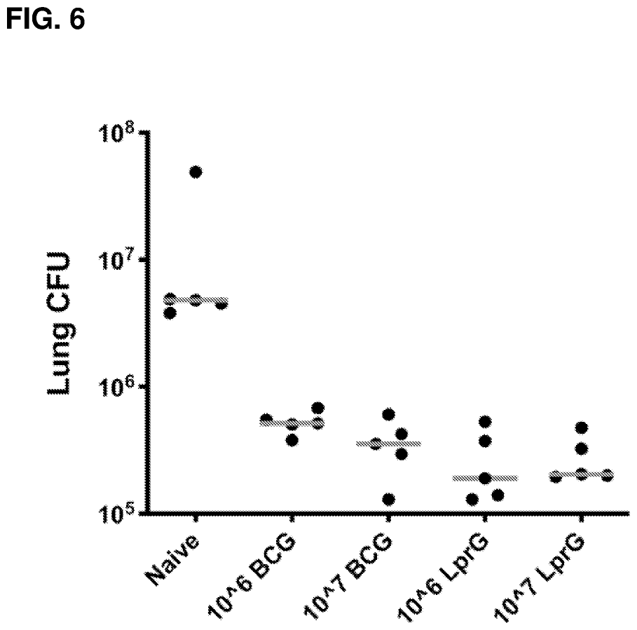

C3HeB/FeJ mice vaccinated with the ΔLprG vaccine demonstrated a median 1.3 log₁₀ reduction in bacterial CFU in lung (FIG. 5A) and a 1.2 median log₁₀ reduction in bacterial CFU in spleen (FIG. 5B) as compared with unvaccinated (Naïve) mice. Although the ΔLprG vaccine resulted in equivalent protection to BCG in C57BL/6J and Balb/cJ mice, the ΔLprG vaccine showed better protection than BCG in C3HeB/FeJ mice with a median 0.9 log₁₀ greater reduction in CFU in lung (FIG. 5A; p<0.05). BCG afforded less protection in C3HeB/FeJ mice as compared with C57BL/6J and Balb/cJ mice (FIG. 1). Dose finding studies in C57BL/6J mice suggested that challenge dose was unlikely to contribute to the improved protective efficacy seen in C3HeB/FeJ mice vaccinated with the ΔLprG vaccine, since 1 log lower vaccine dose (10⁶ CFU versus 10⁷ CFU) showed only minimal differences in protective efficacy for BCG or ΔLprG in C57BL/6J mice (FIG. 6).

At necropsy, ΔLprG and BCG vaccinated mice had fewer and smaller granulomas as compared to naïve mice (FIG. 5C), but ΔLprG vaccinated animals had significantly fewer granulomas with acid-fast bacteria (FIG. 5D), consistent with the overall decreased bacterial burden compared to lungs from BCG vaccinated and unvaccinated animals. Unvaccinated mice had multiple granulomas >1 mm² in area (FIGS. 5C, 5E) with neutrophilic infiltrates and necrosis (FIG. 5E), as compared to BCG and ΔLprG vaccinated mice (FIGS. 5F, 5G). Pathology in non-vaccinated mice was characterized by multibacillary proliferation of acid-fast bacteria within macrophages (FIG. 5H, inset), whereas ΔLprG vaccination was associated with formation of granulomas with few to no acid-fast bacteria surrounded by infiltrates of lymphocytes (FIG. 5I, inset).

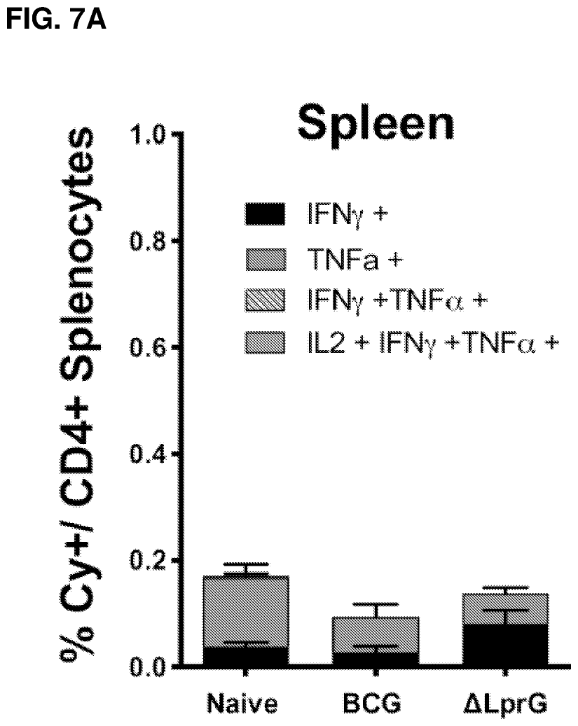

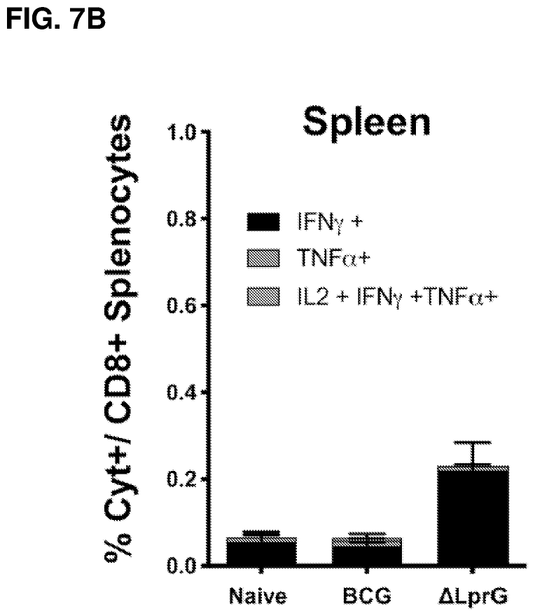

ΔLprG vaccinated mice showed greater Ag-specific CD4⁺ T cell responses in lung than did BCG vaccinated mice and unvaccinated animals at necropsy post-Mtb challenge (FIG. 5J; p=0.027 and p=0.013, respectively). In contrast, no differences were observed in Ag-specific CD8⁺ T cell responses in lung post-challenge across groups (FIG. 5K). CD4⁺ and CD8⁺ T cells responses in spleen post-challenge did not substantially differ between groups (FIGS. 7A-7B). These data suggest that induction of CD4⁺ T lymphocytes in lungs of ΔLprG vaccinated mice contributed to the improved protection in C3HeB/FeJ mice. Indeed, reduction in bacterial CFU in lung correlated with both TNF-α- (FIG. 5L; p=0.001) and IFN-γ/TNF-α-secreting CD4⁺ T cells in lung (FIG. 5M; p=0.007).

### Example 2. Use of IL-17A as a Biomarker for Responsiveness to ΔLprG Administration

ΔLprG Vaccination is Associated with Decreased PD-1 Expression on Antigen Specific T Cells and Correlates with Improved Bacterial Control after Mtb Challenge

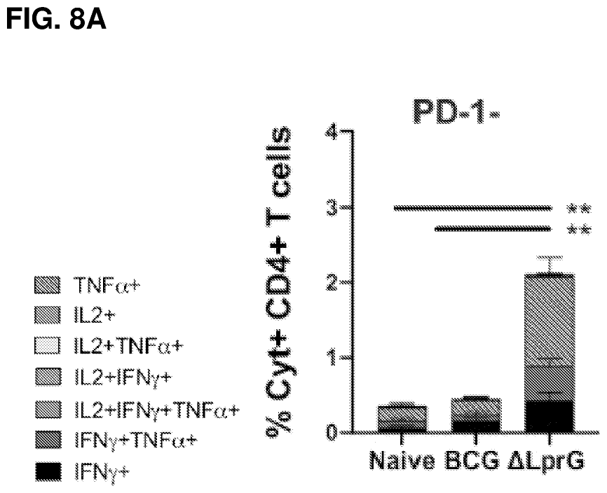

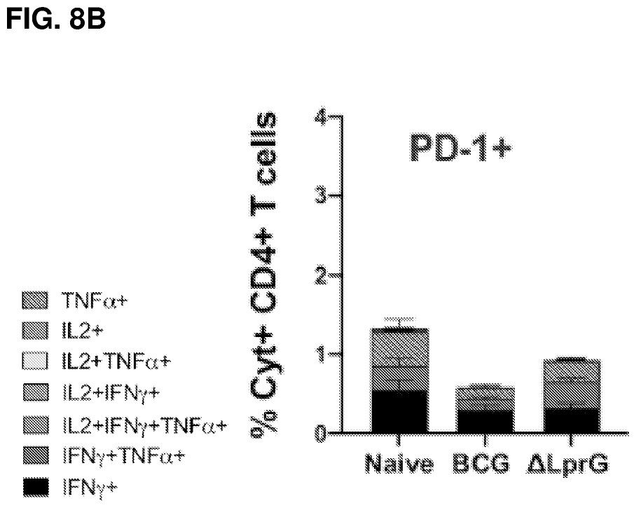

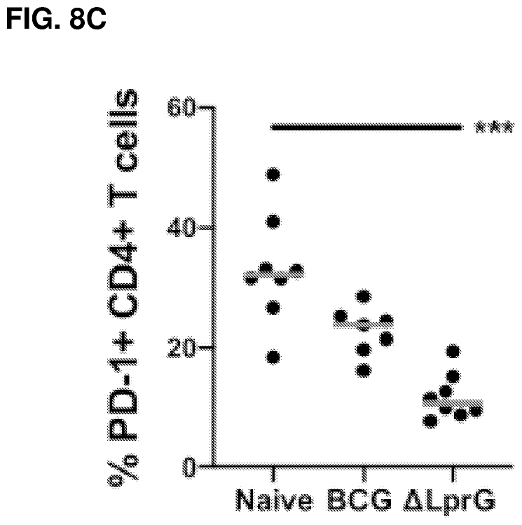

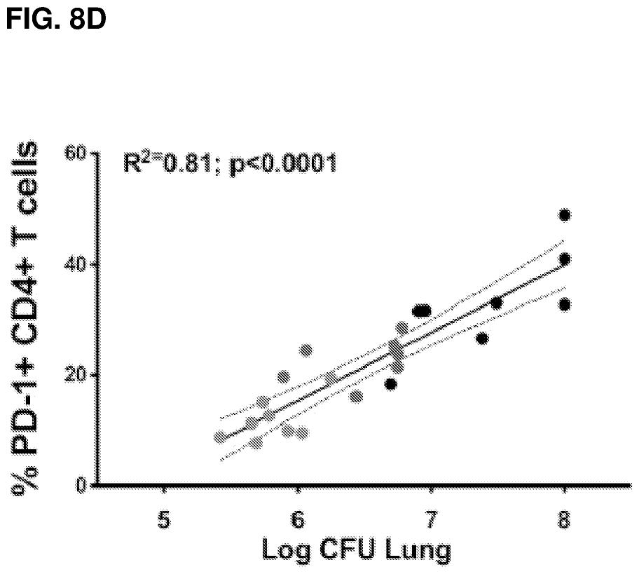

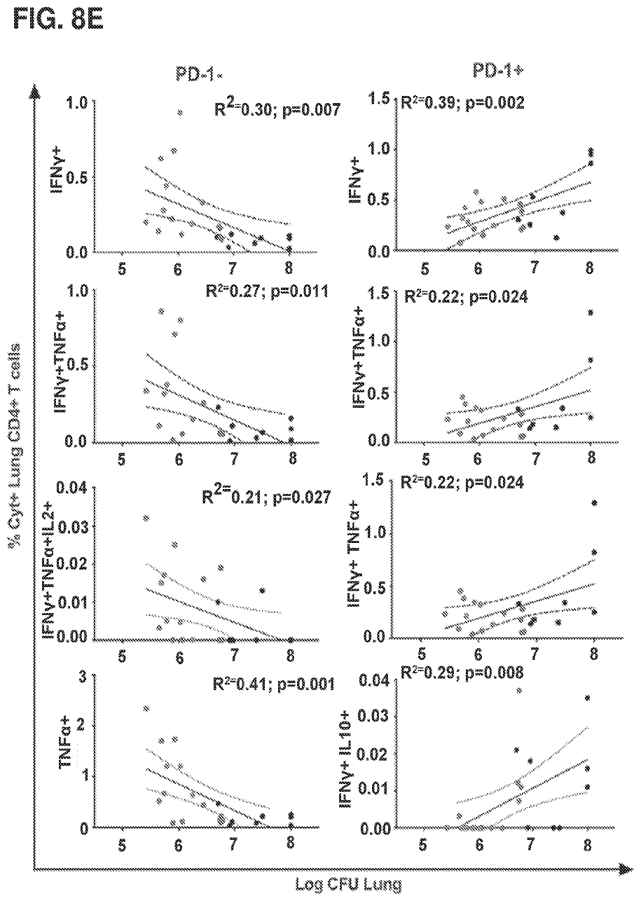

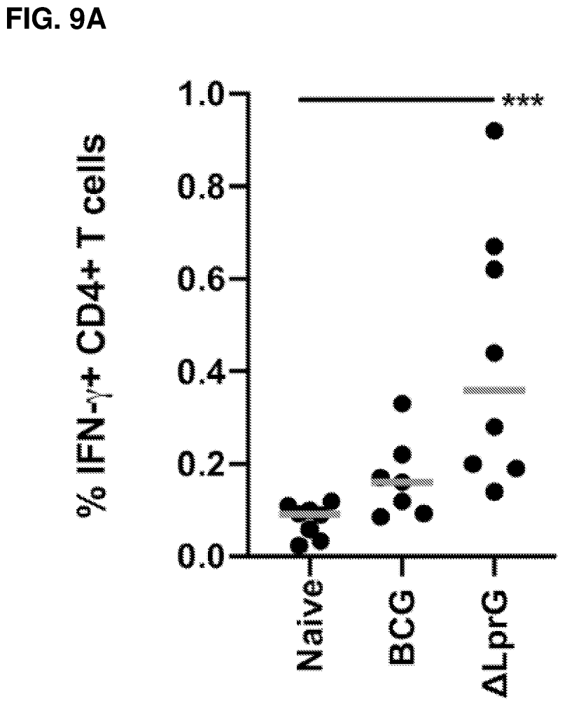

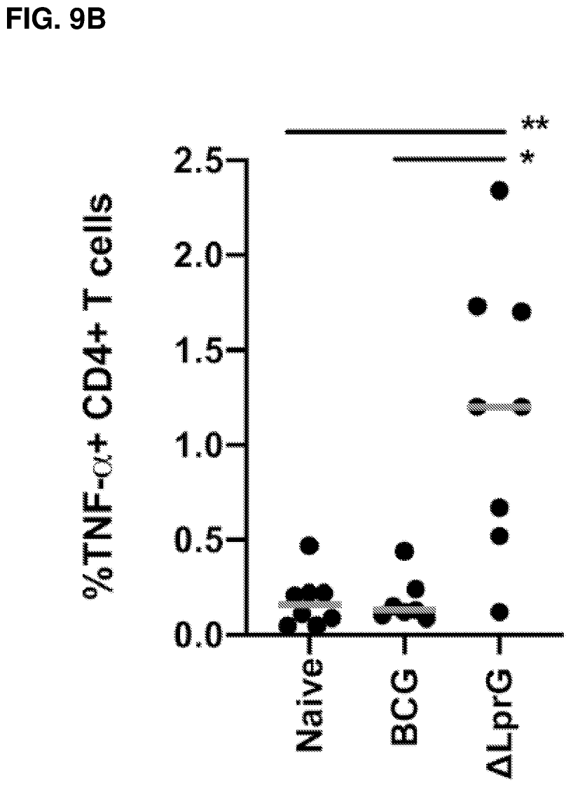

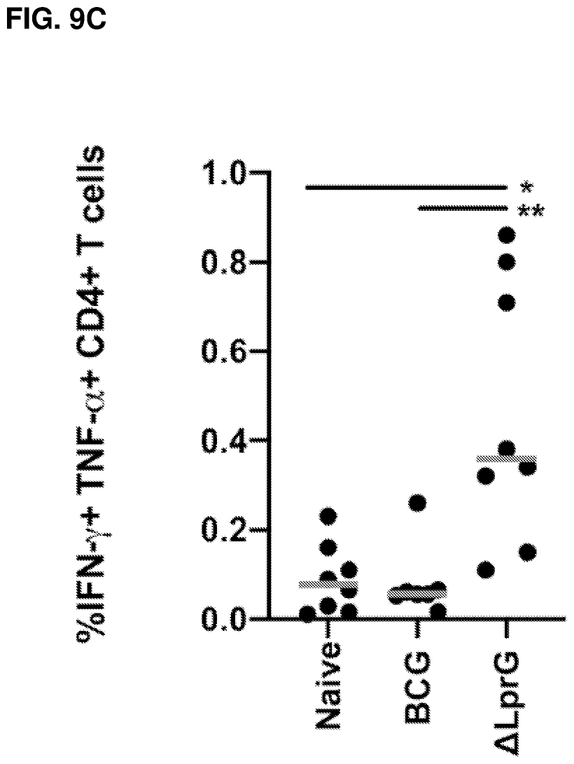

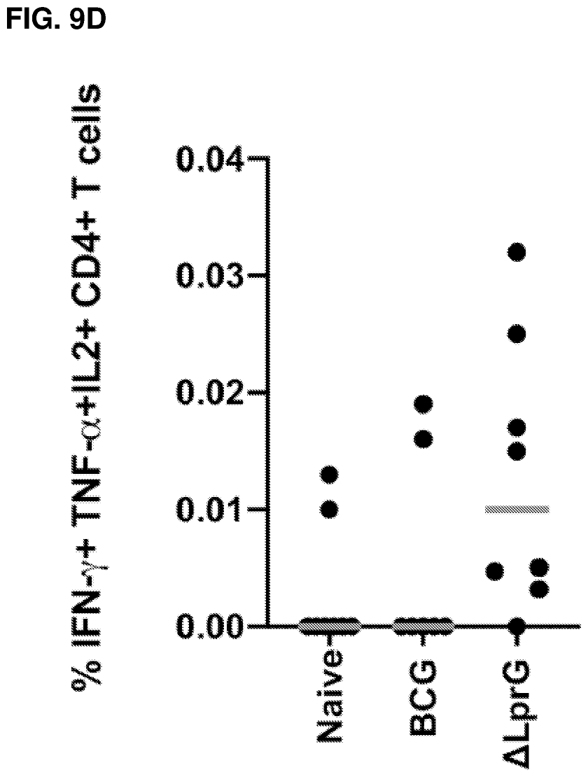

Waning of BCG-induced immunity in humans has been reported to be associated with functionally exhausted effector T-lymphocytes (Andersen and Woodworth. Trends Immunol. 35, 387-395 (2014); Orme. Tuberculosis (Edinb). 90, 329-332 (2010)). PD-1 expression has also been linked to chronic antigenic stimulation and T cell exhaustion during Mtb infection (Jayaraman et al. PLoS Pathog. 12, e1005490 (2016)). We therefore assessed PD-1 expression on Ag-specific cytokine-secreting (Cyt+) CD4⁺ T lymphocytes from lungs of mice at week 4 following Mtb challenge. ΔLprG vaccination resulted in higher PD-1-negative cytokine-secreting Ag-specific CD4⁺ T cell responses in lung than did BCG vaccination or no vaccination (FIG. 8A; p=0.002 to p=0.006; FIGS. 9A-D). In contrast, PD-1-positive cytokine-secreting Ag-specific CD4⁺ T cell responses in lung did not differ across groups (FIG. 8B) despite variation in bacterial burden across groups (FIG. 5A). Prior studies have shown that cells upregulate PD-1 as antigen-experienced cells transition from a central memory to effector state (Crawford et al. Immunity. 40, 289-302 (2014)). PD-1-positive cytokine-negative T lymphocytes may represent exhausted T cells (Wherry and Kurachi. Nat Rev Immunol. 15, 486-499 (2015)). ΔLprG vaccination was associated with significantly lower percentages of cytokine-negative PD-1-positive T cells in lung, reflecting reduced bacterial burden (FIG. 8C) and cytokine-negative PD-1-positive CD4⁺ T-lymphocytes in lung correlated with bacterial CFU in lung following challenge (FIG. 8D; p<0.0001). Among cytokine-secreting Ag-specific CD4+ T cell subsets, PD-1-negative Ag-specific CD4⁺ T cells in lung correlated with decreased bacterial burden (FIG. 8E, left), whereas PD-1-positive Ag-specific CD4⁺ T cells in lung correlated with increased bacterial burden (FIG. 8E, right), suggesting the importance of PD-1-negative memory T cells for protection against Mtb (Wherry and Kurachi. Nat Rev Immunol 15, 486-499 (2015)).

ΔLprG Vaccine Efficacy Correlates with Serum IL-17A Levels Post-Vaccination in C3Heb/FeJ Mice

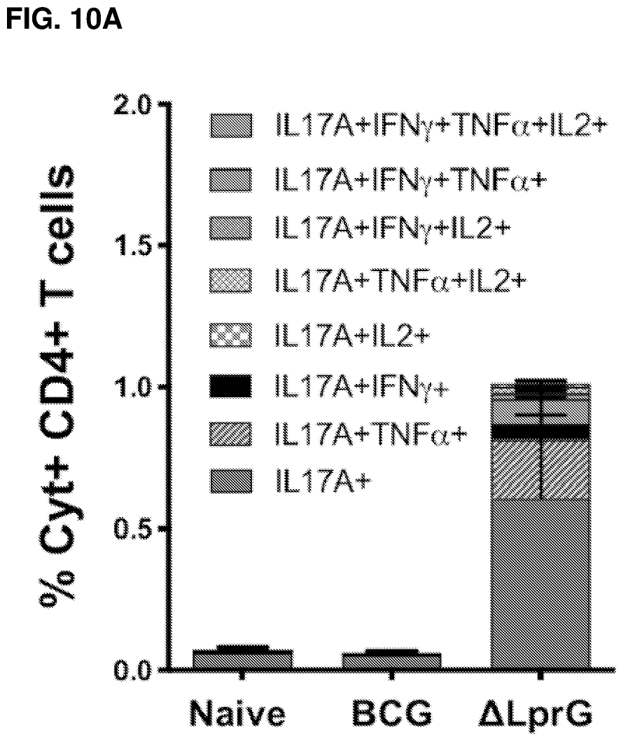

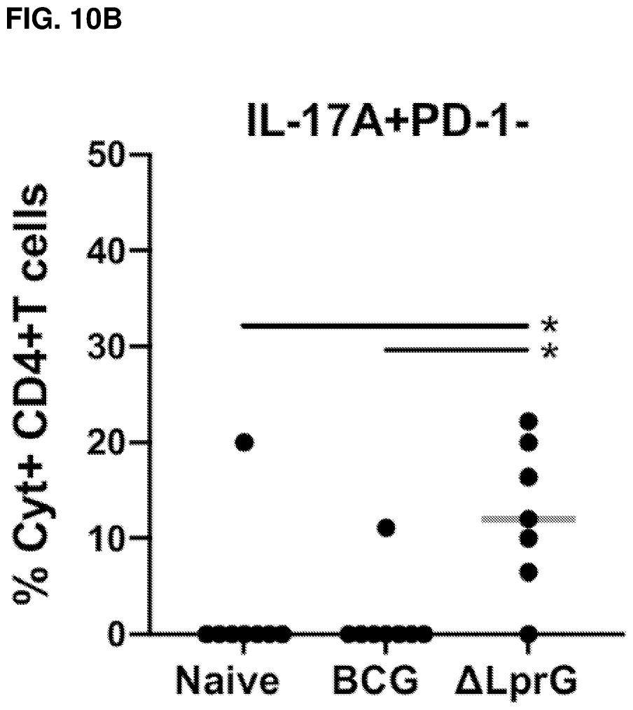

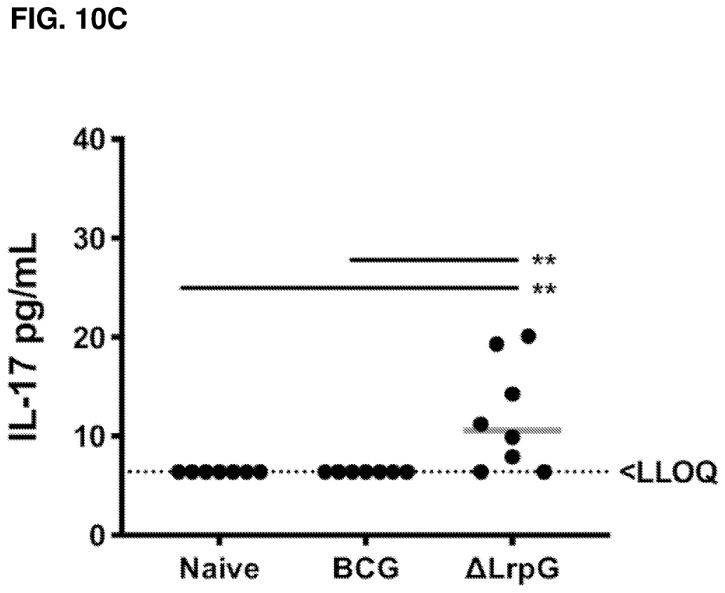

The efficacy of BCG has also been hypothesized to involve IL-17 secreting Ag-specific CD4⁺ T cells (Th17) cells in lung (Gopal et al. Eur. J. Immunol. 42, 364-373 (2012); Khader et al. Nat Immunol. 8, 369-377 (2007)), and expansion of Th17 cells in lung has been linked to improved vaccine-outcomes for experimental TB vaccines (Moliva et al. Mucosal Immunol. 12, 805-815 (2019); Van Dis et al. Cell Rep. 23, 1435-1447 (2018)). Genome-wide association studies have also linked polymorphisms in IL-17 regulatory genes to poor TB clinical outcomes in patients (Wang et al. Scientific Reports. 6, 28586 (2016)) (Zhao et al. Lung. 194, 459-467 (2016)). IL-17+ cells were below the limit of detection in lung and spleen pre-challenge (post-vaccination), and we therefore assessed the expansion of polyfunctional IL-17 secreting CD4+ T cells across vaccine groups following Mtb challenge. IL-17A-secreting Ag-specific CD4⁺ T cell populations were increased in ΔLprG vaccinated mice post-challenge compared with naïve or BCG vaccinated mice (FIG. 10A). Moreover, ΔLprG vaccinated mice demonstrated significantly higher PD-1-negative IL-17A-secreting CD4⁺ T cells in lung compared to naïve and BCG vaccinated mice (FIG. 10B, p=0.013 and 0.020, respectively).

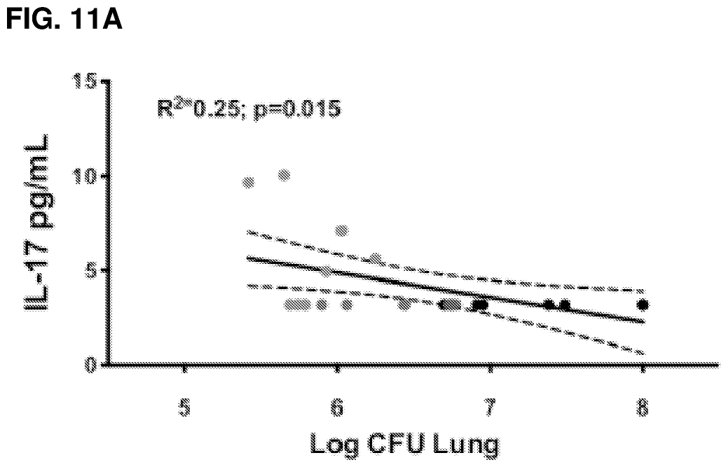

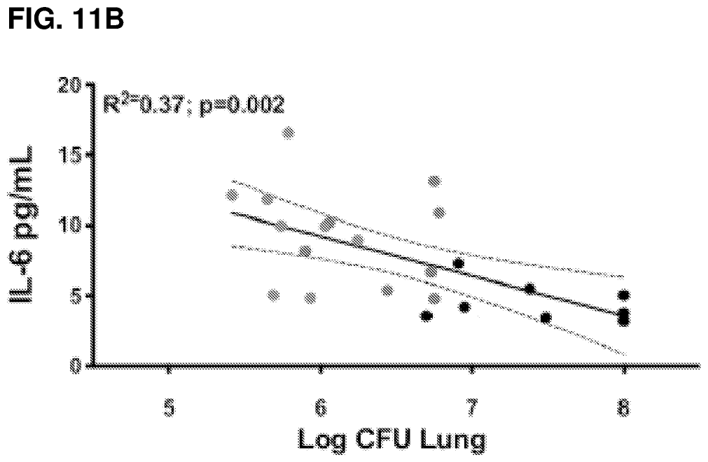

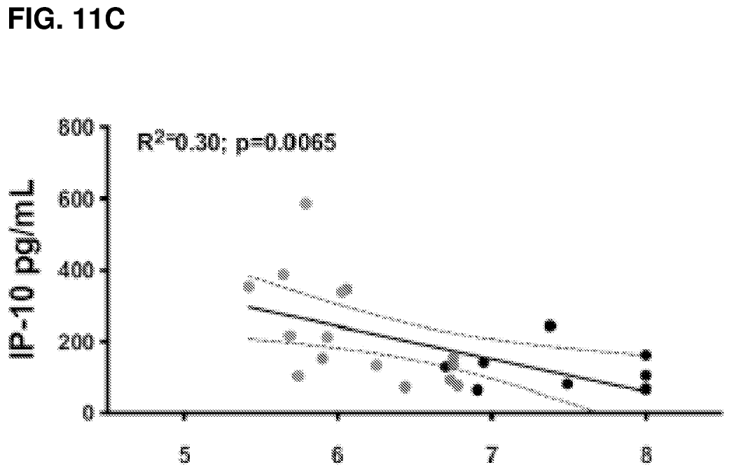

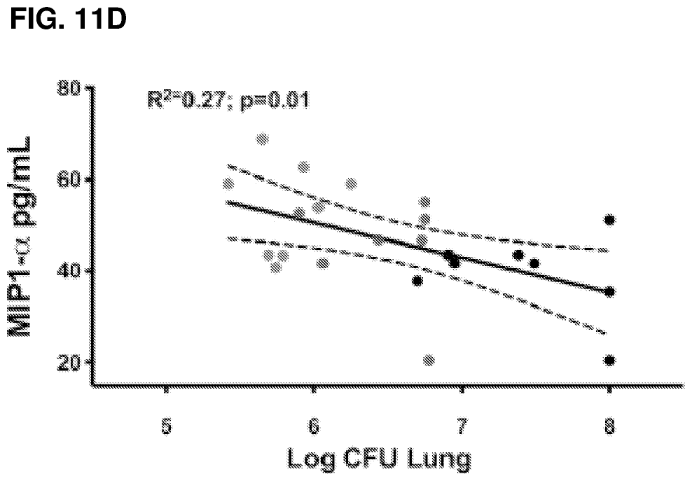

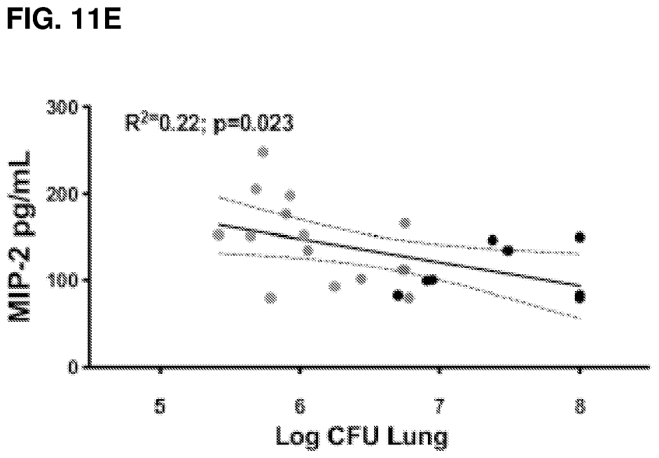

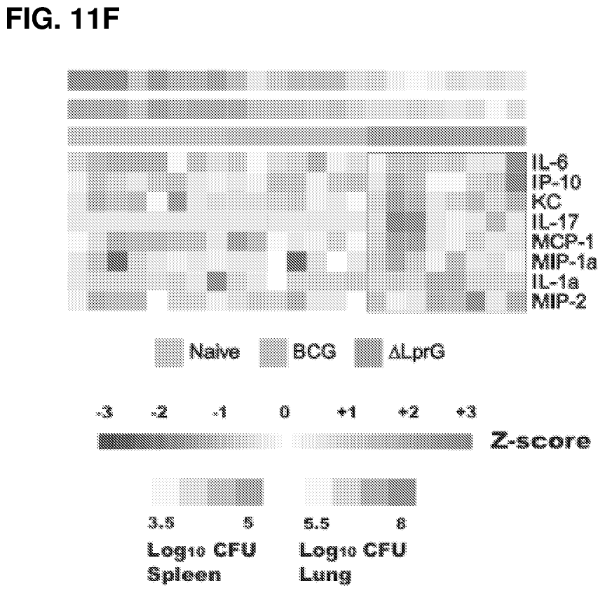

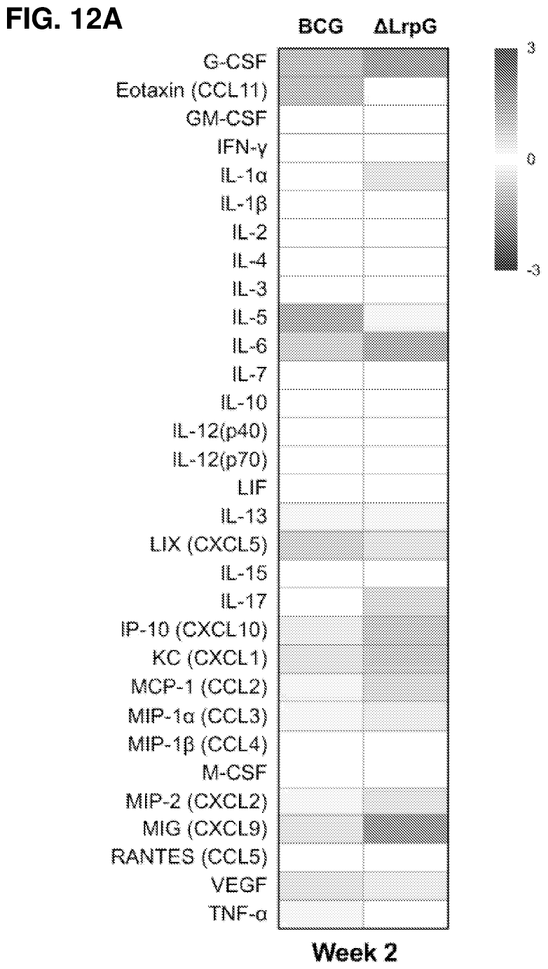

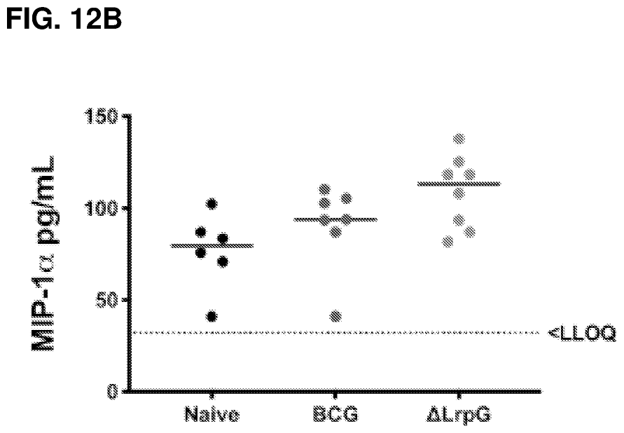

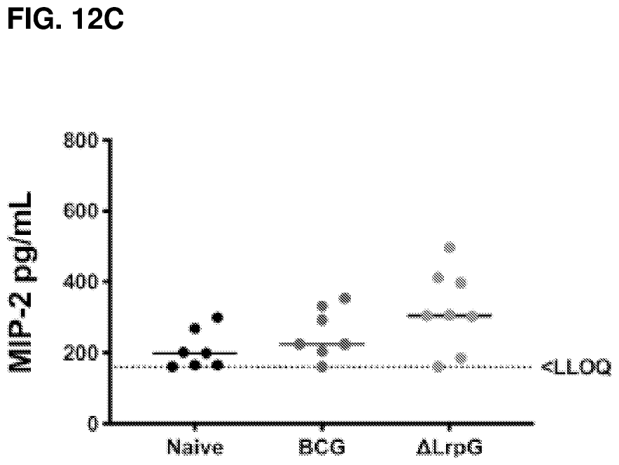

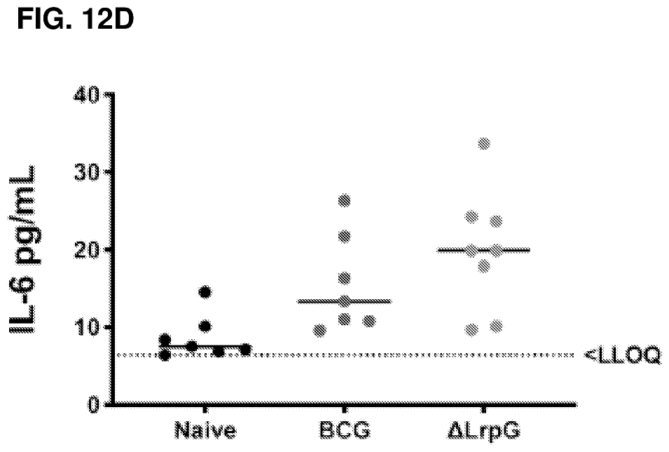

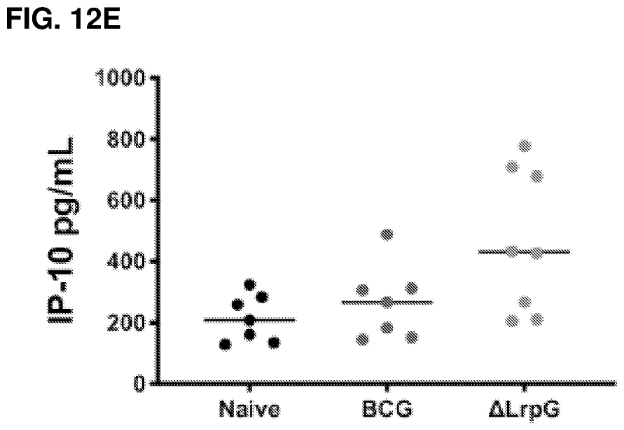

We then determined whether serum cytokine screening can be used to detect early induction of a Th17 cytokine signature during peak immunogenicity post-vaccination, thereby functioning as an early screen for vaccine efficacy in C3HeB/FeJ mice. Consistent with these data, serum IL-17A at week 2 following vaccination was elevated in ΔLprG vaccinated mice as compared to naïve and BCG-vaccinated mice (FIG. 10C, p=0.0053 and 0.0053, respectively). Serum IL-17A levels at week 2 following vaccination correlated with IL-17A-secreting CD4⁺ T cells in lungs following challenge (Table 1). Indeed, elevated IL-17A levels in serum 2 weeks post-vaccination significantly correlated with reduced lung bacterial burden 4 weeks post-Mtb challenge (FIG. 11A; p=0.015). IL-6, IL-1β, and IL-23 have also been reported as Th17 polarizing cytokines (Khader et al. Nat Immunol. 8, 369-377 (2007)). IL-6, MIP-1α, MIP-2, and IP-10, were also elevated in BCG and ΔLprG vaccinated mice (FIGS. 12A-12E) and individually correlated with decreased bacterial burden in lungs following challenge (FIGS. 11B-11E; p=0.002 to p=0.02). To determine if a combination of cytokines would be a better correlate of protection, we used a multiple linear regression analysis to develop a serum cytokine signature that included IL-17A, IL-6, MIP-1α and IP-10a, which correlated with CFU levels in both lung (p=0.008) and in spleen (p=0.02) following challenge (FIG. 11F). However, this combined cytokine signature did not outperform individual cytokines alone, specifically IL-6 and IL-17A, in correlating with protection (Table 2).

TABLE 1
Correlations of Serum IL-17A, Lung
Th17, and Lung Bacterial Burden

| Lung T cell | Lung Th17/Lung CFU | | Serum IL-17/Lung Th17 | |
|---|---|---|---|---|
| cytokine secretion* | R square | P value | R square | P value |
| IL2⁺ IL10⁺ IFNγ⁺ | 0.10 | 0.1389 | 0.34 | 0.0033 |
| TNFα⁺ | | | | |
| IL2⁺ IL17A⁺ IFNγ⁺ | 0.18 | 0.0408 | 0.29 | 0.0085 |
| TNFα⁺ | | | | |
| IL2⁺ IL17A⁺ IFNγ⁺ | 0.10 | 0.1389 | 0.34 | 0.0033 |
| IL2⁺ IL17A⁺ TNFα⁺ | 0.12 | 0.1075 | 0.39 | 0.0015 |
| IL2⁺ IL17A⁺ | 0.10 | 0.1389 | 0.34 | 0.0033 |
| IL2⁺ IFNγ⁺ TNFα⁺ | 0.12 | 0.1053 | 0.16 | 0.0574 |
| IL2⁺ IFNγ⁺ | 0.04 | 0.3726 | 0.00 | 0.9343 |
| IL2⁺ TNFα⁺ | 0.10 | 0.1389 | 0.34 | 0.0033 |
| IL2⁺ | 0.02 | 0.4871 | 0.01 | 0.6160 |
| IL10⁺ IFNγ⁺ TNFα⁺ | 0.06 | 0.2602 | 0.01 | 0.6543 |
| IL10⁺ IFNγ⁺ | 0.26 | 0.013 | 0.01 | 0.6591 |
| IL10⁺ | 0.12 | 0.1129 | 0.02 | 0.5627 |
| IL17A⁺ IFNγ⁺ TNFα⁺ | 0.23 | 0.0207 | 0.37 | 0.0020 |
| IL17A+ IFNγ⁺ | 0.15 | 0.0635 | 0.47 | 0.0003 |
| IL17A⁺ TNFα⁺ | 0.13 | 0.094 | 0.41 | 0.0009 |
| IL17A⁺ | 0.18 | 0.0437 | 0.69 | <0.0001 |
| IFNγ⁺ TNFα⁺ | 0.30 | 0.0067 | 0.06 | 0.2407 |
| IFNγ⁺ | <0.01 | 0.7799 | 0.01 | 0.6394 |
| TNFα⁺ | 0.40 | 0.0012 | 0.41 | 0.0010 |

*Combinations detected by ICS
Table 1. Correlations of % IL-17A+ CD4+ T cells in lung with post-vaccination serum IL-17A levels and lung bacterial burden. Naïve, BCG, or ΔLprG vaccinated mice were challenged with 75 CFU Mtb H37Rv. Serum cytokine levels from naïve and vaccinated mice were assessed at week 2 after vaccination by Luminex assays. Mice were challenged with 75 CFU Mtb H37Rv. Mice were sacrificed at week 4 following challenge and perfused with sterile saline prior to tissue harvest. T cells in lung were collected and stimulated with PPD. Pearson's correlations of % cytokine+ CD4+ T cells with serum IL-17A levels following vaccination and with CFU in lung are shown, and p-values.

TABLE 2
Serum cytokine correlations with bacterial CFU

| Cytokines | p-value Lung | p-value Spleen |
|---|---|---|
| IL-17A | 0.01 | 0.02 |
| IL-6 | 0.002 | 0.004 |
| IP-10 | 0.006 | 0.008 |
| MIP-2 | 0.02 | n.s. |
| MIP-1a | 0.01 | 0.03 |
| MCP-1 | n.s. | 0.02 |
| IL-17A⁺ IL-6⁺ MIP-1a⁺ IP-10 | 0.008 | 0.02 |

Table 2. Correlations of serum cytokine levels following vaccination with CFU following challenge. P values of correlations of serum cytokine and chemokine levels at week 2 following vaccination with CFU levels in lung and spleen following challenge.

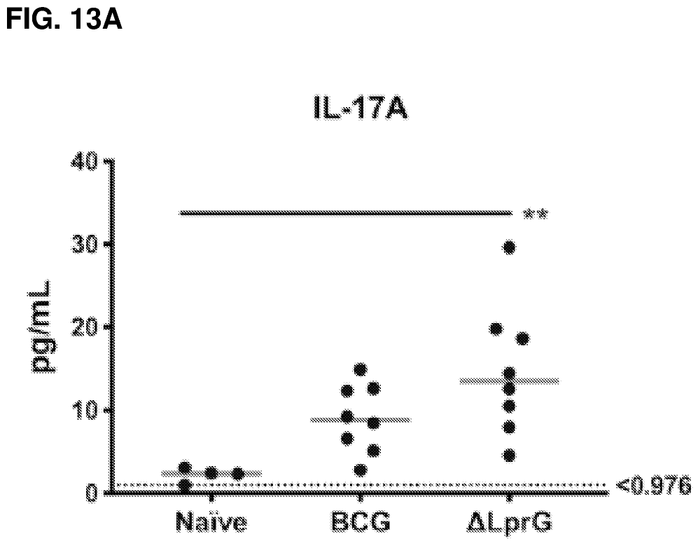

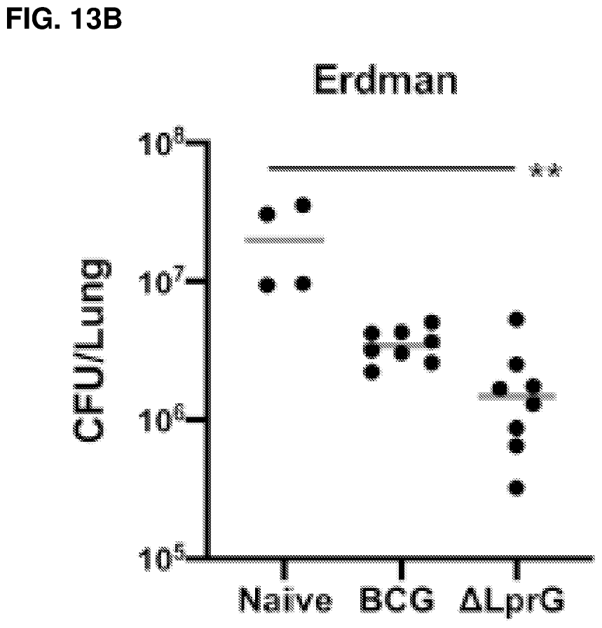

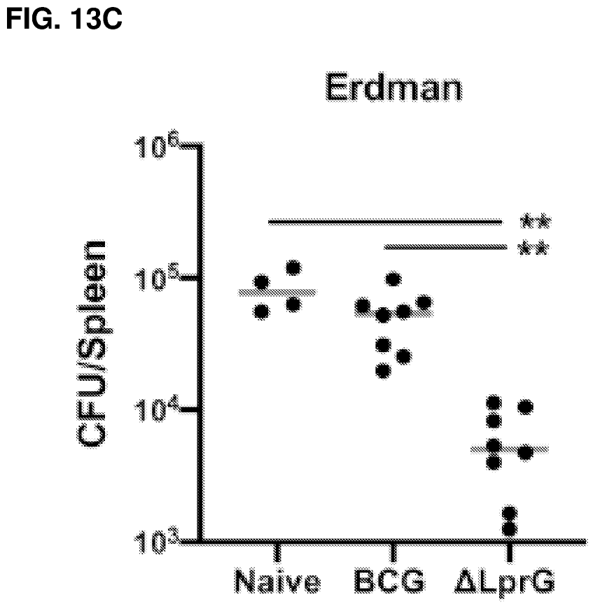

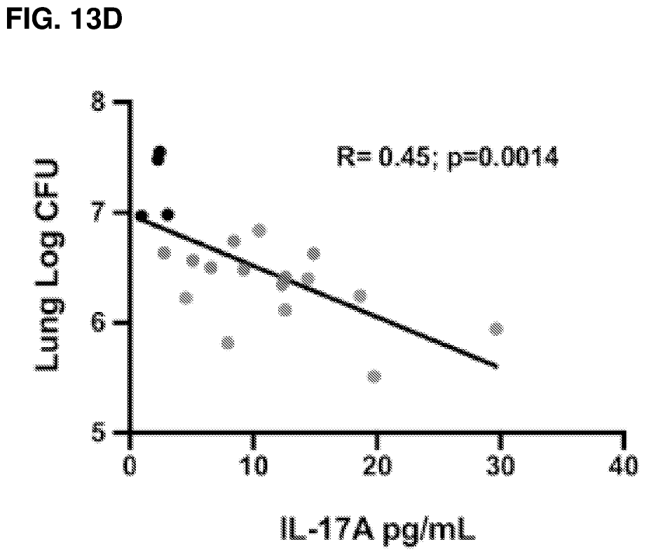

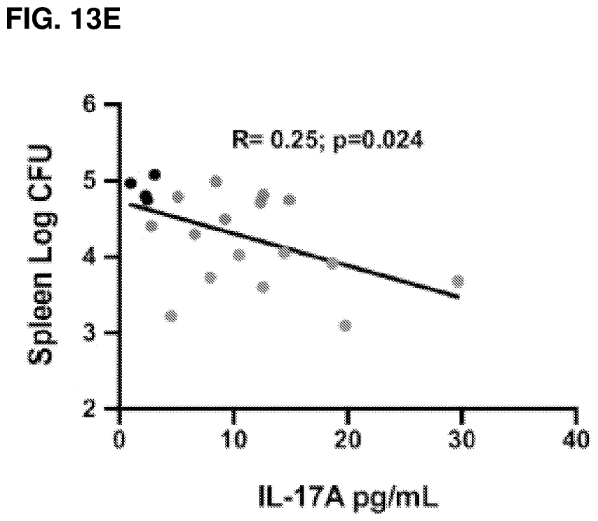

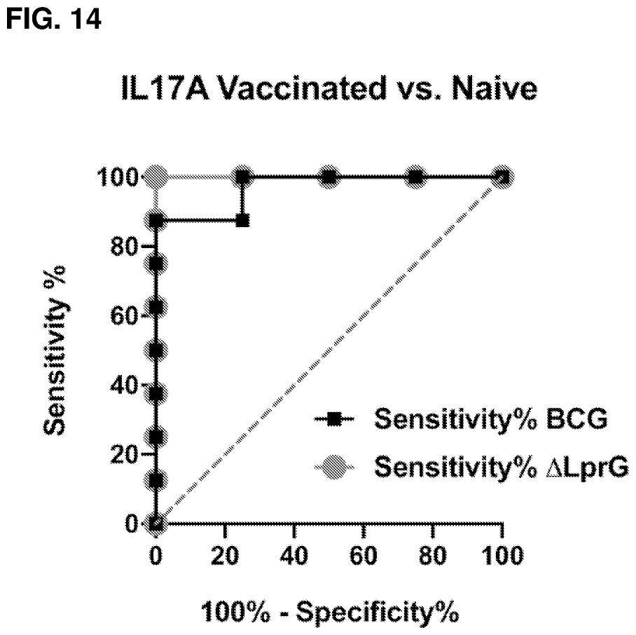

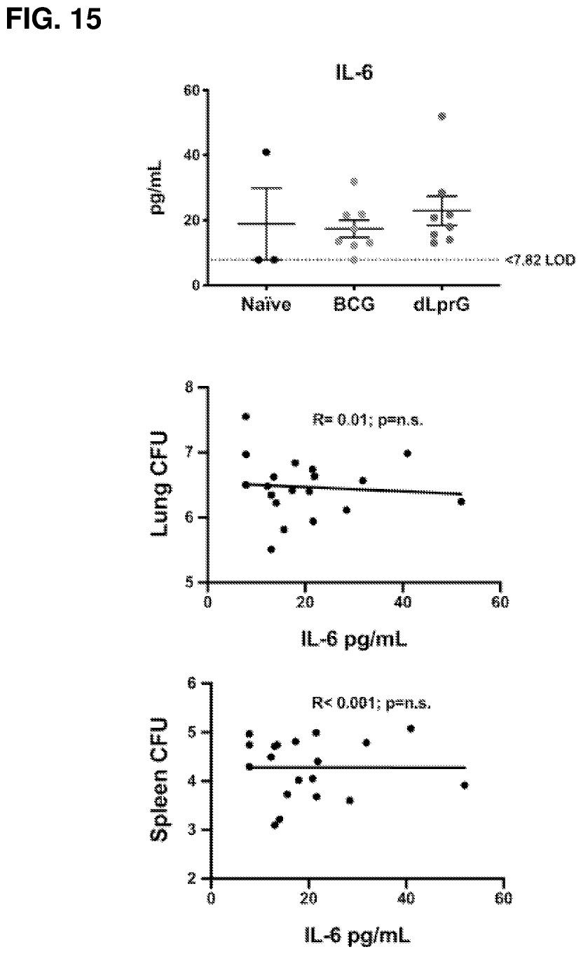

We next assessed the protective efficacy of the ΔLprG vaccine and the predictive capacity of serum IL-17A levels against challenge with the Mtb Erdman strain. In animal models such as the rabbit which demonstrate necrotizing granulomas with Mtb infection, Erdman is considered more pathogenic (Manabe et al. Infect Immun 71, 6004-6011 (2003)), and thus Mtb Erdman is generally considered a stringent challenge strain for vaccine studies. Furthermore, the genetic deletion of the LprG-Rv1410 operon was on the H37Rv background, and we wanted to test efficacy with a challenge strain different from that used to generate the ΔLprG vaccine. We vaccinated mice as described above and used a high sensitivity IL-17A Luminex panel to better quantify IL-17A levels in sera at week 2 following vaccination. Mice were then challenged with the Mtb Erdman strain. ΔLprG and BCG vaccination led to consistent induction of serum IL-17A two weeks following vaccination (FIG. 13A), but ΔLprG afforded greater protection against Mtb Erdman challenge with a median 1.1 log reduction in bacterial CFU in lung (FIG. 13B; p=0.001) and a median 1.2 log reduction in bacterial CFU in spleen (FIG. 13C; p=0.002) compared to unvaccinated controls. In contrast, BCG afforded only a modest reduction in CFU in lung and spleen in this stringent challenge model. Serum IL-17A levels at week 2 following vaccination inversely correlated with bacterial burden in both lung (FIG. 11D, p=0.0014) and spleen (FIG. 11E, p=0.024) with a ROC AUC between 0.97-1.0 (FIG. 14) Yu et al. (2018), supra). In contrast, levels of IL-6 levels failed to correlate with protection against Mtb Erdman challenge (FIG. 15). These data suggest that serum IL-17A is a useful early biomarker following vaccination to predict protective efficacy.

### Example 3. Use of an Immunogenic Composition of ΔLprG Mtb to Treat an Infection

An infection (e.g., a bacterial infection, such as an infection with Mtb) in a subject (e.g., a human) may not induce an immune response to a level sufficient to clear the infection in a timely manner or at all. For instance, a course of an infection may range from 1 to 72 hours, 3 to 7 days, 1 to 8 weeks, 2 to 12 months, 1 to 10 years or longer, or it may persist for the lifetime of the subject. In this instance, an immunogenic composition of ΔLprG Mtb, as described herein, can be administered to the subject to treat or reduce the term of the infection.

### Example 4. Use of IL-17A as a Biomarker for Vaccine Responsiveness

Vaccination induces heterogeneous immune responses and the magnitude of the responses can be insufficient to confer the desired level of protective immunity (e.g., sterilizing immunity). As a result, multiple doses of a vaccine are often administered to increase the likelihood of inducing the desired level of protective immunity. Furthermore, variety in a population's responsiveness can make the responsiveness of an individual to a particular dose of a vaccine uncertain.

IL-17A can be used as a biomarker to monitor responsiveness of an immune system in a subject to a vaccine or other immunogenic composition, such as ΔLprG Mtb, BCG, MTBVAC, VPM1002, DAR-901, MVA85A, ChAdOx1.PPE15, TB/FLU-04L, Ad5Ag85A, AERAS-402, M72 (e.g., M72/AS01_{E}), RUTI, H107, or CysVac2/Advax, in which an increase in IL-17A in a sample from a subject over a baseline level (e.g., a level present in a sample taken from the subject prior to administration of the vaccine or immunogenic composition) or over a reference level (e.g., a level present in a healthy individual or a level present in a responsive individual) identifies the subject as responsive to the vaccine or immunogenic composition. A subject that exhibits an IL-17A level that is less than a baseline level or a reference level indicates that the vaccine or immunogenic composition may not have been sufficient to stimulate a protective immune response in the subject. Consequently, re-administration of the vaccine or immunogenic composition or administration of a different vaccine or immunogenic composition may be warranted.

### Example 5. Use of an Immunogenic Composition of ΔLprG Mtb in a Prime-Boost Immunization

Protective immune responses induced by immunogenic compositions often wane to below a protective level over time. As a result, re-administration of an immunogenic composition or administration of a different immunogenic composition is necessary to maintain a protective immune response.

An immunogenic composition of ΔLprG Mtb can be administered to a subject which has previously been administered an immunogenic composition containing one or more Mtb antigens (e.g., BCG, MTBVAC, VPM1002, DAR-901, MVA85A, ChAdOx1.PPE15, TB/FLU-04L, Ad5Ag85A, AERAS-402, M72 (e.g., M72/AS01_{E}), RUTI, H107, or CysVac2/Advax). In this instance, the ΔLprG Mtb is administered as a boost immunization in a prime-boost immunization strategy.

The timing of administration of the boost immunization can be a pre-set time (e.g., 1, 2, 3, or more weeks, 1, 2, 3, 4, 5, 6, 7, 8, 9, 10, 11, or more months, or 1, 2, 3, 4, 5, 6, 7, 8, 9, 10, or more years after administration of the prime immunization) or a time determined by measurement of one or more biomarkers (e.g., one or more of the biomarkers described herein (e.g., IL-17A)).

For example, a boost immunization of ΔLprG Mtb may be administered to a subject soon (e.g., 1, 2, or 3 weeks, 1, 2, 3, 4, 5, 6, 7, 8, 9, 10, or 11 months, or 1 or 2 years) after a prime immunization if a sample from the subject has a level of IL-17A determined to be below a reference level or may be administered at a later time (e.g., 3, 4, 5, 6, 7, 8, 9, 10, or more years) after a prime immunization if a sample from the subject has a level of IL-17A determined to be above a reference level.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A pharmaceutical composition comprising between 1×10² CFU and 1×10¹⁰ CFU of a mycobacterium tuberculosis strain comprising one or more mutations that ablate or reduce expression of LprG and Rv1410 (ΔLprG Mtb) in a volume of between 0.05 mL and 3 mL.

2. The pharmaceutical composition of claim 1, wherein the ΔLprG Mtb is live or whole cell or is inactivated by heat, fixation, or radiation.

3. The pharmaceutical composition of claim 1 or 2, further comprising a pharmaceutically acceptable vehicle, diluent, and/or excipient.

4. The pharmaceutical composition of any one of claims 1-3, further comprising an adjuvant.

5. The pharmaceutical composition of any one of claims 1-4, wherein the pharmaceutical composition is in a form suitable for subcutaneous, intradermal, intravenous, intramuscular, transdermal, parenteral, intranasal, respiratory, perioral, sublingual, oral, or topical administration.

6. The pharmaceutical composition of any one of claims 1-5, wherein the composition is in lyophilized, solid or liquid form.

7. The pharmaceutical composition of any one of claims 1-6, wherein the ΔLprG Mtb further comprises one or more mutations that ablate or reduce expression of one or more additional genes.

8. The pharmaceutical composition of claim 7, wherein at least one additional gene is selected from the group containing fad26, phoP, sigH, pan, and leu.

9. The pharmaceutical composition of any one of claims 1-8, wherein the ΔLprG Mtb encodes one or more transgenes.

10. The pharmaceutical composition of claim 9, wherein at least one transgene encodes a cytokine, a chemokine, an immunoregulatory agent, or a therapeutic agent.

11. The pharmaceutical composition of claim 9 or 10, wherein at least one transgene contains a foreign antigen.

12. The pharmaceutical composition of any one of claims 1-11, wherein the composition is capable of inducing an immune response in a human.

13. The pharmaceutical composition of any one of claims 1-12, wherein the composition is a vaccine.

14. A method of inducing an immune response in a human comprising administering the pharmaceutical composition of any one of claims 1-13 to the human.

15. The method of claim 14, wherein administering the pharmaceutical composition treats or prevents a disease.

16. The method of claim 15, wherein administration of the pharmaceutical composition:
i) reduces the symptoms of a disease;
ii) prevents the reemergence of a disease from latency;
iii) reduces sequela of a disease; and/or
iv) reduces the transmissibility of a disease.

17. The method of claim 14 or 15, wherein the disease is an infectious disease.

18. The method of claim 17, wherein the infectious disease is caused by one or more bacteria.

19. The method of claim 18, wherein one or more bacteria are Mycobacterium spp.

20. The method of claim 19, wherein at least one Mycobacterium spp. is selected from M. tuberculosis, M. leprae, M. bovis, M. africanum, M. avium, M. canetti, M. chelonae, M. fortuitum, M. gordonae, M. hiberniae, M. intracellulare, M. kansasii, M. marinum, M. microti, M. paratuberculosis, M. phlei, M. pinnipedii, M. scrofulaceum, M. simiae, M. smegmatis, M. szulgai, M. ulcerans, M. vacca, and M. xenopi.

21. The method of claim 20, wherein at least one Mycobacterium spp. is M. tuberculosis.

22. The method of any one of claims 14-21, wherein the composition is administered as a single dose.

23. The method of any one of claims 14-21, wherein the composition is administered as a plurality of doses.

24. The method of claim 23, wherein the doses are administered at least one day apart.

25. The method of claim 23, wherein said plurality of doses are administered at least two weeks apart.

26. The method of claim 23, wherein the composition is administered twice.

27. The method of any one of claims 14-26, wherein the composition is delivered by subcutaneous, intradermal, intravenous, intramuscular, transdermal, parenteral, intranasal, respiratory, perioral, sublingual, oral, or topical administration.

28. The method of any one of claims 14-27, wherein the composition is administered as either a priming component or a boosting component in a prime-boost immunization.

29. The method of claim 28, wherein the composition is administered as the priming component and the boosting component is selected from a whole cell vaccine, a recombinant vector vaccine, or a subunit vaccine.

30. The method of claim 29, wherein the whole cell vaccine is selected from BCG, MTBVAC, VPM1002, or DAR-901.

31. The method of claim 29, wherein the recombinant vector vaccine is selected from MVA85A, ChAdOx1.PPE15, TB/FLU-04L, Ad5Ag85A, or AERAS-402.

32. The method of claim 29, wherein the subunit vaccine is selected from M72, RUTI, H107, or CysVac2/Advax.

33. The method of claim 28, wherein the composition is administered as the boosting component and the priming component is selected from a whole cell vaccine, a recombinant vector vaccine, or a subunit vaccine.

34. The method of claim 33, wherein the whole cell vaccine is selected from BCG, MTBVAC, VPM1002, or DAR-901.

35. The method of claim 33, wherein the recombinant vector vaccine is selected from MVA85A, ChAdOx1.PPE15, TB/FLU-04L, Ad5Ag85A, or AERAS-402.

36. The method of claim 33, wherein the subunit vaccine is selected from M72, RUTI, H107, or CysVac2/Advax.

37. A method of monitoring responsiveness of a subject to an immunogenic composition that has been administered for treatment or prevention of an infection, comprising detecting a level of IL-17A in a sample from the subject that is obtained after administration of the immunogenic composition, wherein detection of said level of IL-17A in the sample that is higher than a reference level identifies the subject as responsive to the treatment and a level that is lower than or equal to the reference level identifies the subject as unresponsive to the treatment.

38. The method of claim 37, wherein the immunogenic composition is a vaccine.

39. The method of claim 37 or 38, wherein the reference level of IL-17A is the level of IL-17A present in a sample from the subject prior to administration of the immunogenic composition.

40. The method of claim 37, wherein the reference level of IL-17A is a level of IL-17A present in the 5^{th} percentile of a reference population.

41. The method of claim 37, wherein the reference level of IL-17A is a level of IL-17A present in the 50^{th} percentile of a reference population.

42. The method of claim 37, wherein the reference level of IL-17A in a sample is the level of IL-17A present in the 95^{th} percentile of a reference population.

43. The method of any one of claims 37-42, wherein the level of IL-17A is detected between 1 minute and 12 weeks after administration of the immunogenic composition to the subject.

44. The method of any one of claims 37-43, wherein the infection is a bacterial infection.

45. The method of claim 44, wherein the bacterial infection is an infection by one or more Mycobacterium spp.

46. The method of claim 45, wherein at least one Mycobacterium spp. is selected from M. africanum, M. avium, M. bovis, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi.

47. The method of claim 46, wherein at least one Mycobacterium spp. is M. tuberculosis.

48. The method of any one of claims 37-47, wherein the immunogenic composition is a M. tuberculosis immunogenic composition or vaccine.

49. The method of claim 48, wherein the immunogenic composition or vaccine comprises the composition of any one of claims 1-13 alone or in combination with one or more of BCG, MTBVAC, VPM1002, DAR-901, MVA85A, ChAdOx1.PPE15, TB/FLU-04L, Ad5Ag85A, AERAS-402, M72, RUTI, H107, or CysVac2/Advax.

50. The method of any one of claims 37-49, wherein the sample is blood; optionally serum or plasma.

51. The method of any one of claims 37-50, wherein the subject is a mammal.

52. The method of claim 51, wherein the mammal is a human.

53. A kit comprising a composition of any one of claims 1-13 and a reagent for measuring a level of IL-17A in a sample.

54. The kit of claim 53, wherein the kit further comprises one or more of BCG, MTBVAC, VPM1002, DAR-901, MVA85A, ChAdOx1.PPE15, TB/FLU-04L, Ad5Ag85A, AERAS-402, M72, RUTI, H107, or CysVac2/Advax.

55. The kit of claim 53 or 54, wherein the reagent is an immunoassay reagent.

56. The kit of claim 55, wherein the reagent is for use in an ELISA.

57. The kit of any one of claim 55 or 56, wherein the sample is blood; optionally serum or plasma.

58. The kit of any one of claims 55-57, wherein the kit further comprises instructions for use.

59. The kit of any one of claims 55-58, wherein the kit further comprises one or more samples comprising a known amount of IL-17A.

60. The pharmaceutical composition of claim 1, further comprising an adjuvant.

61. The pharmaceutical composition of claim 1, wherein the pharmaceutical composition is in a form suitable for subcutaneous, intradermal, intravenous, intramuscular, transdermal, parenteral, intranasal, respiratory, perioral, sublingual, oral, or topical administration.

62. The pharmaceutical composition of claim 1, wherein the composition is in lyophilized, solid or liquid form.

63. The pharmaceutical composition of claim 1, wherein the ΔLprG Mtb further comprises one or more mutations that ablate or reduce expression of one or more additional genes.

64. The pharmaceutical composition of claim 1, wherein the ΔLprG Mtb encodes one or more transgenes.

65. The pharmaceutical composition of claim 64, wherein at least one transgene encodes a cytokine, a chemokine, an immunoregulatory agent, or a therapeutic agent.

66. The pharmaceutical composition of claim 64, wherein at least one transgene contains a foreign antigen.

67. The pharmaceutical composition of claim 1, wherein the composition is capable of inducing an immune response in a human.

68. The pharmaceutical composition of claim 1, wherein the composition is a vaccine.

69. A method of inducing an immune response in a human comprising administering the pharmaceutical composition of claim 1 to the human.

70. The method of claim 69, wherein administering the pharmaceutical composition treats or prevents a disease.

71. The method of claim 70, wherein administration of the pharmaceutical composition:
i) reduces the symptoms of a disease;
ii) prevents the reemergence of a disease from latency;
iii) reduces sequela of a disease; and/or
iv) reduces the transmissibility of a disease.

72. The method of claim 69, wherein the disease is an infectious disease.

73. The method of claim 72, wherein the infectious disease is caused by one or more bacteria.

74. The method of claim 73, wherein one or more bacteria are Mycobacterium spp.

75. The method of claim 74, wherein at least one Mycobacterium spp. is selected from M. tuberculosis, M. leprae, M. bovis, M. africanum, M. avium, M. canetti, M. chelonae, M. fortuitum, M. gordonae, M. hiberniae, M. intracellulare, M. kansasii, M. marinum, M. microti, M. paratuberculosis, M. phlei, M. pinnipedii, M. scrofulaceum, M. simiae, M. smegmatis, M. szulgai, M. ulcerans, M. vacca, and M. xenopi.

76. The method of claim 75, wherein at least one Mycobacterium spp. is M. tuberculosis.

77. The method of claim 69, wherein the composition is administered as a single dose.

78. The method of claim 69, wherein the composition is administered as a plurality of doses.

79. The method of claim 78, wherein the doses are administered at least one day apart.

80. The method of claim 78, wherein said plurality of doses are administered at least two weeks apart.

81. The method of claim 78, wherein the composition is administered twice.

82. The method of claim 69, wherein the composition is delivered by subcutaneous, intradermal, intravenous, intramuscular, transdermal, parenteral, intranasal, respiratory, perioral, sublingual, oral, or topical administration.

83. The method of claim 69, wherein the composition is administered as either a priming component or a boosting component in a prime-boost immunization.

84. The method of claim 83, wherein the composition is administered as the priming component and the boosting component is selected from a whole cell vaccine, a recombinant vector vaccine, or a subunit vaccine.

85. The method of claim 84, wherein the whole cell vaccine is selected from BCG, MTBVAC, VPM1002, or DAR-901.

86. The method of claim 84, wherein the recombinant vector vaccine is selected from MVA85A, ChAdOx1.PPE15, TB/FLU-04L, Ad5Ag85A, or AERAS-402.

87. The method of claim 84, wherein the subunit vaccine is selected from M72, RUTI, H107, or CysVac2/Advax.

88. The method of claim 83, wherein the composition is administered as the boosting component and the priming component is selected from a whole cell vaccine, a recombinant vector vaccine, or a subunit vaccine.

89. The method of claim 88, wherein the whole cell vaccine is selected from BCG, MTBVAC, VPM1002, or DAR-901.

90. The method of claim 88, wherein the recombinant vector vaccine is selected from MVA85A, ChAdOx1.PPE15, TB/FLU-04L, Ad5Ag85A, or AERAS-402.

91. The method of claim 88, wherein the subunit vaccine is selected from M72, RUTI, H107, or CysVac2/Advax.

92. The method of claim 37, wherein the level of IL-17A is detected between 1 minute and 12 weeks after administration of the immunogenic composition to the subject.

93. The method of claim 37, wherein the infection is a bacterial infection.

94. The method of claim 93, wherein the bacterial infection is an infection by one or more Mycobacterium spp.

95. The method of claim 94, wherein at least one Mycobacterium spp. is selected from M. africanum, M. avium, M. bovis, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi.

96. The method of claim 95, wherein at least one Mycobacterium spp. is M. tuberculosis.

97. The method of claim 37, wherein the immunogenic composition is a M. tuberculosis immunogenic composition or vaccine.

98. The method of claim 97, wherein the immunogenic composition or vaccine comprises the composition of claim 1 alone or in combination with one or more of BCG, MTBVAC, VPM1002, DAR-901, MVA85A, ChAdOx1.PPE15, TB/FLU-04L, Ad5Ag85A, AERAS-402, M72, RUTI, H107, or CysVac2/Advax.

99. The method of claim 37, wherein the sample is blood; optionally serum or plasma.

100. The method of claim 37, wherein the subject is a mammal.

101. The method of claim 100, wherein the mammal is a human.

102. A kit comprising a composition of claim 1 and a reagent for measuring a level of IL-17A in a sample.

103. The kit of claim 102, wherein the kit further comprises one or more of BCG, MTBVAC, VPM1002, DAR-901, MVA85A, ChAdOx1.PPE15, TB/FLU-04L, Ad5Ag85A, AERAS-402, M72, RUTI, H107, or CysVac2/Advax.

104. The kit of claim 102, wherein the reagent is an immunoassay reagent.

105. The kit of claim 104, wherein the reagent is for use in an ELISA.

106. The kit of claim 104, wherein the sample is blood; optionally serum or plasma.

107. The kit of claim 104, wherein the kit further comprises instructions for use.

108. The kit of claim 104, wherein the kit further comprises one or more samples comprising a known amount of IL-17A.
